(19) United States
(12) Patent Application Publication
(10) Pub. No.: US 2003/0027330 A1
(43) Pub. Date: Feb. 6, 2003

(54) Method for facilitating the production of differentiated cell types and tissues from embryonic and adult pluripotent and multipotent cells

(76) Inventors: Robert Lanza, Clinton, MA (US); Michael West, Boston, MA (US)

Correspondence Address: Pillsbury Winthrop LLP, Intellectual Property Group, 1600 Tysons Boulevard, McLean, VA, 22102, US

(21) Appl. No.: 10/112,939

(22) Filed: Apr. 2, 2002

Related U.S. Application Data
(60) Provisional application No. 60/280,138, filed on Apr. 2, 2001.

(51) Int. Cl. C12N 5/08; C12N 15/85

(52) U.S. Cl. 435/366; 435/455

(57) ABSTRACT

The invention is concerned with producing differentiated cells, tissues and organs from pluripotent and mutlipotent cells. The methods of the invention are particularly useful for producing differentiated cells from pluripotent cells wherein communication between the cells of more than one embryonic germ layer or more than one organ system are required for development along a specific cell lineage. The invention methods are effected by in vivo or in vitro culturing of embryonic and developing or developed allogeneic or xenogeneic cells.

### CROSS REFERENCE TO RELATED APPLICATION

This application claims priority from U.S. Provisional Application Serial No. 60/280,138 filed Apr. 2, 2001, which is incorporated herein in its entirety.

### FIELD OF INVENTION

The present invention is concerned with developing differentiated cells and tissues from pluripotent and multipotent embryonic or adult stem cells or progenitor cells. In particular, the invention provides methods that facilitate the isolation of particular cell types, especially cells wherein their differentiation is directed by in vivo or in vitro environments requiring interaction between different cells or cell lineages. The methods are useful for generating replacement cells and tissues for transplantation, and for assisting in treatments geared toward the regeneration of diseased or injured tissues.

### BACKGROUND OF THE INVENTION

The developmental processes that govern the ontogeny of multicellular organisms, including humans, hinge on the interplay between signaling pathways and the natural communications between cells. The process of embryogenesis gradually narrows the developmental potential of cells as development proceeds from the original totipotent fertilized egg to the terminally differentiated mature cell. These terminally differentiated cells have specialized functions and characteristics, and represent the last step in a multi-step process of precursor cell differentiation into a particular cell type.

Gastrulation, the morphogenic movement of the early embryonic cell mass, results in the formation of three distinct germ cell layers, the ectoderm, the mesoderm, and the endoderm. As cells in each germ cell layer respond to various developmental signals, specific organs and cavities are generated which are composed of specific differentiated cells. Although it is common to classify particular cell types in terms of the embryonic layer from which they arise, differentiation does not result in the constituent cells of layers being so separate as to completely diverge in subsequent development. In fact, during subsequent development of the various organ systems, derivatives of the different layers are often closely interlocked and interdependent in terms of fundamental morphogenesis. Gray's Anatomy, 37th ed., ed. Williams et al., 1989.

Nevertheless, for convenience, the contributions of the three different layers may be generalized as follows. The primitive embryonic ectoderm, for instance, gives rise to, among others, the epidermis, the lining of the cells of the neighboring glands, the appendages of the skin, hair and nails, the nervous system, including the cranial and spinal ganglia, the neuroepithelium of the sense organs, some salivary glands and the enamel of the teeth, and epithelial linings of the anal canal and the male and female genitalia. The ectoderm is also divided into separate subregions including the general body ectoderm, the neural plate, the neural crest and the ectodermal plactodes. For a more complete description of which cell types arise from each of the subregions, see Gray's Anatomy, supra, herein incorporated by reference for its analysis of embryogenesis.

The primitive embryonic endoderm gives rise to the epithelial lining of the whole of the alimentary canal, the linings cells of the glands which open into it, including the liver and the pancreas and their ducts, the epithelial lining of the auditory tube and tympanic cavity, the epithelium of the thyroid and parathyroid glands and the thymus, the lining epithelium of the larynx, trachea and smaller air passages including the alveoli and air saccules, the epithelium of most of the urinary bladder and much of the urethra, and the epithelium of the prostate and many other paraurethral glands. In particular, pancreatic islet cells are thought to be endodermal in origin.

The primitive intraembryonic mesoderm gives rise to the remaining organs and tissues of the body, including all connective and sclerous tissues, the teeth with the exception of the enamel, the whole musculature of the body, including the striated and unstriated muscle, the blood, vasculature, lymph and lymphatic systems, the urogenital system except most of the lining of the bladder, prostate and urethra, the cortex of the suprarenal glands and the mesothelial linings of the pericardial, pleural and peritoneal cavities. In all vertebrate embryos, the mesoderm becomes incompletely divided by a longitudinal groove into the paraxial part and the lateral plate, with the groove separating these sections, or the intermediate mesoderm, subsequently developing into the nephrogenic cord and thereafter into the renal corpuscles, nephric tubules, the ureter and renal tubules in both sexes, the whole of the gonadal tissues except for the sex cells, and mesenteries and connective framework of all of the foregoing among others. The paraxial mesoderm thereafter undergoes a segmentation process, resulting in the mesodermal somites which eventually form the vertebrae and associated joints and ligaments. The lateral plate mesoderm is split by the intraembryonic coelom into somatic and splanchnic layers, with the somatic mesothelial lining forming the pericardium and peritoneum, and the splanchopleuric mesenchymal cells later differentiating into the muscles, blood vessels, lymphatics, adipose and connective tissues of the walls of the heart and gastrointestinal tract.

Notwithstanding the convenient classification of various organs and differentiated cells as being endodermal, mesodermal or ectodermal in origin, it is clear that intricate interplay between various intercellular signaling events guides the development of non-terminally differentiated precursor cells and ultimately dictates specific cellular identities. To a large degree, organ formation depends on the interactions between mesenchymal cells with the adjacent epithelium. The formation of the limbs, the gut organs, e.g., liver or pancreas, kidney, teeth, etc., all depend on interactions between specific mesenchymal and epithelial components. In fact, the differentiation of a given epithelium depends on the nature of the adjacent mesenchyme. For example, when lung bud epithelium is cultured alone, no differentiation occurs. However, when lung bud epithelium is cultured with stomach mesenchyme or intestinal mesenchyme, the lung bud epithelium differentiates into gastric glands or villi, respectively. Further, if lung bud epithelium is cultured with liver mesenchyme or bronchial mesenchyme, the epithelium differentiates into hepatic cords or branching bronchial buds, respectively. See U.S. Pat. No. 6,149,902, herein incorporated by reference in its entirety.

Despite the recognition of the interplay between the three embryonic layers during cellular differentiation and organogenesis, the art is void of methodology that seeks to produce differentiated cells and organs from specific pluripotent and multipotent stem and precursor cells by exposing such cells to cell mixtures and embryonic structures that mimic the embryonic environment and facilitate cell differentiation. For instance, U.S. Pat. No. 5,639,618 of Gay describes a method whereby pluripotent embryonic stem cells are transfected with a construct comprising the regulatory region of a lineage specific gene operably linked to a DNA encoding a reporter protein, the pluripotent stem cell is then permitted to differentiate randomly, and the cells expressing the reporter protein are separated from the other cells by virtue of the reporter protein. However, such an approach is less than ideal for obtaining human differentiated cells from pluripotent stem cells, given the risk of forming an embryo and the ethical considerations associated therewith.

Also the prior art methods are problematic because they may induce genetic modifications, the results of which are uncertain and pose regulatory and safety concerns, particularly if the cells are to be used for human cell therapy. Additionally, the presence and expression of transgenes in the cells may result in rejection upon transplantation into an allogeneic host.

Similarly, U.S. Pat. No. 5,733,727 of Fields describes the isolation of cardiomyocytes following the in vitro differentiation of embryonic stem cells that had been transfected with a selectable marker, whereby the selectable marker permits the isolation of the cells away from cells of other lineages. Fields also suggests obtaining the skeletal myoblasts or cardiomyocyte grafts by introducing myogenic precursor cells into the myocardial tissue of a living animal, however, such random differentiation in vitro accompanied by in vivo exposure to formed organs to facilitate graft production may not enable the isolation of all desirable cell types, particularly those which require the interaction and cross-signaling of cells in more than one embryo layer to receive the proper developmental cues.

U.S. Pat. No. 5,942,225 of Bruder et al describes the lineage-directed induction of human mesenchymal stem cell differentiation by exposing such stem cells to a bioactive factor or combination of factors effective to induce differentiation either ex vivo or in vivo, wherein the bioactive factor is described as a morphogenetic factor or cytokine that induces differentiation along a desired developmental path. However, it is not suggested that such stem cells be exposed to an embryonic environment or structure or combination of cells that would give the necessary inductive signals for differentiation of many cell types, therefore this method will be limited to the isolation of cells for which the specific protein messengers required for differentiation have been identified.

Researchers have shown using an in utero xenotransplantation approach that neural progenitor cells from mice differentiate into cells having glial-like features after injection into the rat forebrain ventricle. See Winkler et al, June 1998, “Incorporation and glial differentiation of mouse EGF-responsive neural progenitor cells after transplantation into the embryonic rat brain,” Neurosci. 11(3): 99-116. Similarly, human neural precursor cells that had been expanded in vitro were shown to develop into neurons in a site-specific manner after being transplanted into either an adult or neonatal rat brain. See Fricker et al, July 1999, “Site-specific migration and neuronal differentiation of human neural progenitor cells after transplantation into the adult rat brain,” J. Neurosci. 12(7): 2405-13. In these studies, however, the resulting neuron cells were not purified but were rather traced by mouse-specific and human-specific markers. Recently, many such reports of successful transplant of xenogeneic cells and migration to appropriate sites and differentiation have been called into question. In any event, such approaches are not likely to result in the production of formed tissues, or in the isolation of cells the development of which requires cross-signaling between different layers of the developing embryo.

Thus, there is a need for methods that facilitate the development of replacement cells of any desired type, particularly cell types which form in the context of embryogenesis, and in the context of cross-signaling between the three layers of the embryo.

### BRIEF DESCRIPTION OF INVENTION

The present invention solves the helps solve the deficiencies of the prior art by providing a method whereby the proper environmental cues encountered in the process of cellular differentiation and organogenesis are employed to facilitate the production of specific differentiated cell types and tissues from embryonic and adult pluripotent cells. The methods reported herein are particularly useful for obtaining desired mammalian cell types the development of which requires the interaction of several cell types, indeed, possibly even the interaction of all three germ layers.

In the case of generating human replacement cells/tissues, it would be ethically problematic to allow inner cell mass (ICM)/embryonic cells to develop to the point where the three germ layers start to interact to generate the structures found in embryos. However, the present invention presents methods whereby human ICM, primordial or pluripotent stem cells are mixed with various formed embryonic structures or developing organ systems, such as human or animal teratomas, teratocarcinomas or other groups or mixtures of embryonic cells or structures, to generate chimeric structures in order to help induce the human cells to develop into the desired replacement cell type. In the case of xenogeneic combinations, these are then implanted or injected into animals that are either immuno-compromised, immuno-suppress or tolerized in order to generate differentiated cells and tissues. Also described are in vitro techniques where human or animal cells are juxta posed with pluripotent stem cells to provide induction of desired differentiation pathways.

Thus the present invention includes methods of producing replacement cells and tissues from pluripotent and adult stem and precursor cells. The invention also encompasses methods of obtaining such cells from the animal host or in vitro environment in which they are developed, as well as methods of using the formed cells and tissues for transplantation and for regenerating injured tissues in a patient in need thereof.

### BRIEF DESCRIPTION OF THE FIGURES

FIGS. 1 and 2 show that large discs of bone are obtained on injection of parthenogenically derived stem cells (Cyno-1 stem cells produced by parthenogenic activation of oocytes derived from cynomologus monkeys.

FIG. 3 shows a colony of white blood cells obtained from cells in the liver of a cloned cow.

FIG. 4 shows multiple colonies of red blood cells derived from a single primitive blood cell obtained from the liver of a cloned cow fetus.

FIG. 5 shows cells in the liver of a cloned fetal cow. It can be seen therefrom that most are developing into red blood cells. On average one per thousand cells should be a stem cell.

FIG. 6 shows a primitive blood forming stem cell contained in the liver of a cloned cow fetus.

FIG. 7 shows a colony of stem cells derived from the liver of a cloned cow fetus growing in contact with bone marrow stromal cells.

FIG. 8 contains the results of a polymerase chain reaction (PCR) that detects expression of a Neo marker gene in a cloned fetal cow liver.

FIG. 9 contains the results of a PCR detection assay showing that the Neo gene is detected in peripheral blood of cells following transplantation of fetal liver stem cells from a cloned fetal cow nuclear donor. (The neo gene was also detected in primitive blood progenitor cells using colony assay detection methods).

FIGS. 10 and 11 contain CFC assay results from blood samples derived from a normal cow and cows that were transplanted with HSCs from the liver of a cloned cow fetus

FIG. 12 shows that a pluripotent cynomougous primate ES cell line produced by parthenogenic activation of unfertilized oocytes results in a differentiated cell mixture comprising mesenchymal cells, endothelial cells and myocardial cells juxtaposed to one another.

FIG. 13 shows a tissue culture apparatus system for co-culture of pluripotent cells and endothelial inducer cells.

FIG. 14 shows a tissue culture apparatus system for co-culture of pluripotent cells, endothelial inducer cells, and stromal cell inducer on a polymeric matrix.

### DETAILED DESCRIPTION OF THE INVENTION

The present invention provides methods for promoting or inducing the development of pluripotent or multipotent cells along a particular path of differentiation and development by exposing such cells to an environment conducive to the cellular cross-talk or induction that occurs between multiple cell types and potentially multiple germ layers during embryogenesis. In particular, the invention includes a method of producing differentiated mammalian cells or tissues, comprising:

(a) obtaining a pluripotent or multipotent stem cell;

(b) mixing said pluripotent or multipotent stem cell with developing allogeneic or xenogeneic cells; and

(c) implanting or injecting said mixture of cells into a suitable host embryo, fetus or animal so as to generate differentiated mammalian cells or tissues; or alternatively culturing said cell mixture under conditions conducive for differentiation.

The methods of the invention are useful for obtaining cells and tissues for patients in need of replacement cells and tissues. Preferably, the patients to be treated by the present invention are human patients, but the methods could be employed for obtaining cells and tissues for any mammal, including primates, agricultural animals such as cows and pigs, domestic pets such as cats or dogs, wild animals, including extinct or endangered animals.

The pluripotent or multipotent stem cells used in the methods of the invention may be either embryonic or adult cells. A preferred cell to be used is an inner cell mass (ICM) cell, wherein the ICM cell is obtained following nuclear transfer from a donor cell from the patient in need of replacement cells and tissues. A “pluripotent” cell refers to a cell that is capable of dividing into multiple lineages of cells, but differs from a totipotent cell in that it does not have the capability of generating an entire embryo. For instance, an ES or ICM cell is pluripotent, but being formed from the inner cell mass, would not form the trophectoderm necessary to incase the growing embryo. Therefore, ES cells and ICMs are considered to be pluripotent. Multipotent cells, on the other hand, are non-terminally differentiated precursor cells that are capable of differentiating into a variety of different cell types along a particular lineage, but would not have the full potential of pluripotent cells.

For instance, embryonic pluripotent cells useful in the methods of the invention include primordial germ cells, embryoid body cells, ES cells, ICM cells, blastocyst cells, morula cells, committed progenitor cells, mesenchymal stem cells (MSC), neural crest cells, cranial crest cells. Embryonic cell types may be produced by nuclear transfer such as described in earlier patents assigned to the University of Massachusetts, Roslin Institute and PPL Therapeutics among others. Alternatively, embryonic cells may be derived from parthenogenically produced embryos, both gynogenic or androgenic parthenogenically activated embryos (e.g., by activation of unfertilized ovum), or from embryos produced by IVF procedures. Also pluripotent cells may be derived by prolonged culturing of ICMs on feeder layer cultures. Nuclear transfer embryos include these derived by transplantation or fusion of the same or different species cell, nucleus or chromosomes into a suitable recipient cell, e.g. an oocyte or ES cell which is enucleated prior, concurrent or after transplantation or fusion. For example, human blastocysts may be obtained by implantation or fusion of a human cell, nucleus or chromosomes with a rabbit or bovine oocyte, which is activated. Adult stem cells are stem cells that exist in the adult body that have not terminally differentiated, and include mesenchymal stem cells (MSC), hematopoietic stem cells, stromal stem cells, neural precursor cells, liver precursor cells, skin precursor cells, mesodermal precursor cells, endodermal precursor cells, ectodermal precursor cells among others

A wide variety of pluripotent and multipotent cells are available in the art for use in the present invention, or may be obtained using methods known in the art. For instance, U.S. Pat. No. 5,914,268 of Keller et al provides a method of obtaining an embryonic stem cell-derived pluripotent embryoid body cell population having one or more cells capable of developing into cells of the hematopoietic and/or endothelial lineage and is herein incorporated in its entirety. Shamblott and colleagues disclosed the isolation of human embryonic germ cells through the process of embryoid body formation, and these cells have been shown to have the capability to derive a wide variety of cells in culture. See Shamblott et al, Jan. 2, 2001, “Human embryonic germ cell derivatives express a broad range of developmentally distinct markers and proliferate extensively in vitro,” Proc. Natl. Acad. Sci. USA, 98(1): 113-18. U.S. Pat. No. 5,827,735 of Young et al provides a method of producing purified pluripotent mesenchymal stem cells from muscle, and is herein incorporated by reference in it entirety. U.S. Pat. No. 6,200,206 of Peterson and Nousek-Goebl provides methods for the isolation of hematopoietic precursor cells and is herein incorporated by reference.

As discussed, the pluripotent cells to be used in the methods of the present invention may also be obtained using nuclear transfer technology. Such methods are described in U.S. Pat. No. 5,945,577 to Stice et al., and U.S. Pat. No. 6,147,276 to Wilmut and Campbell, herein incorporated by reference in their entirety. Donor cells may be of any cell cycle, i.e., G1, G2, G0 S or M and may be diploid, haploid or tetraploid. Also, such cells may be obtained by prolonged culturing of inner cell masses in tissue culture to produce stable pluripotent cell lines referred to as CICMS as described in U.S. Pat. No. 5,905,042 or 5,994,619 both incorporated by reference herein in their entirety. These methods are exemplified with ungulate CICMS but may be used with other species ICMS, particularly humans and other primates. This route is particularly useful for transplant patients where suitable pluripotent or multipotent cells cannot be obtained or found in the body, and cells, tissues or organs having immune compatibility are desired. Nuclear transfer is also useful in the context where the patient's own cells suffer from a genetic deficiency or mutation that is able to be corrected prior to tissue production. In such cases, it is possible to insert, delete or correct genetic material using recombinant technology prior to nuclear transfer in order to generate cells, tissues and organs that are free of the mutated DNA.

As used herein, the terms “develop,” “differentiate” and “mature” all refer to the progression of a cell from a stage of having the potential to differentiate into at least two different cell lineages to becoming a specialized or differentiated cell. Such cells may be terminally differentiated, i.e., as would be cells in organs and tissues, or may be non-terminally differentiated as would be the case in obtaining a hematopoietic multipotent stem cell from a pluripotent precursor cell. Preferred cells and tissues produced according to the invention are human cells or tissues, and more specifically are replacement cells or tissues generated for a patient in need thereof.

Any desired replacement cell type may be produced using the methods of the invention. However, the invention is particularly suited for cells which require the interaction of more than one germ layer in order for the precursor pluripotent or multipotent cell to differentiate and generate such cells. For instance, possible replacement cells or tissues that may be obtained by the present methods include pancreatic islet cells, liver cells, kidney cells, lung cells, gut organ tissues, heart muscle cells or other cardiac and vascular tissue, skin cells and other fibroblasts, muscle cells, cells of sensory organs such as the eyes, nose, tongue, ears, hematopoietic cells and cells of the lymph and immune systems, skeletal and cartilage cells, neural cells and tissues, reproduction and endocrine gland cells and tissues, etc. The invention is particularly suitable, however, for cells such as pancreatic islet cells, the development of which requires crosstalk among cells of different germ layers during embryogenesis.

In preferred embodiments, differentiation of embryonic cell types discussed above, e.g. human ICM or ES cells, into different lineages of somatic cells can be effected using the following preferred co-cultures:

i) Differentiation of osteoblasts can be effected by co-culture with dural cells.

ii) Hormonal cocktail, sertoli cells and testicular stromal cells can be used to generate mature sperm.

ii) Differentiation into astrocytes can be effected using endothelial cells

iii) Production of cardiomyocytes can be effected using neonatal rat cardiomyocytes

iv) Generation of Keratinocytes human dermal fibroblasts can be effected by use of dead, de-epidermized human dermis

v) Product of Dopaminergic neurons can be effected using PA6 stromal cells

vi) Production of CD34+CD38-cells can be effected using porcine microvascular endothelial cell layer and a cocktail of FLT3L, SCF, IL-6, and GM-CSF cytokine combination

vii) Primate tissues such as intestine, bone, cartilage, ganglion, hair, hair follicles, etc. using a teratoma cell in SCID mice (See e.g., Spector, J. A. et al. (2002) Co-culture of osteoblasts with immature dural cells causes an increased rate and degree of osteoblast differentiation. Plast Reconstr Surg 109 (2), 631-642; discussion 643-634; Buttery, L. D. et al. (2001) Differentiation of osteoblasts and in vitro bone formation from murine embryonic stem cells. Tissue Eng 7 (1), 89-99; Sousa, M. et al. (2002) developmental potential of human spermatogenetic cells co-cultured with Sertoli cells. Hum Reprod 17 (1), 161-172; Mi, H. et al. (2001) Induction of astrocyte differentiation by endothelial cells. J Neurosci 21 (5), 1538-1547; Condorelli, G. et al. (2001) Cardiomyocytes induce endothelial cells to trans-differentiate into cardiac muscle: implications for myocardium regeneration. Proc Natl Acad Sci U S A 98 (19), 10733-10738; Bagutti, C. et al. (2001) Dermal fibroblast-derived growth factors restore the ability of beta(1) integrin-deficient embryonal stem cells to differentiate into keratinocytes. Dev Biol 231 (2), 321-333; Kawasaki, H. et al. (2000) Induction of midbrain dopaminergic neurons from ES cells by stromal cell-derived inducing activity. Neuron 28 (1), 31-40; Rosler, E. et al. (2000) Cocultivation of umbilical cord blood cells with endothelial cells leads to extensive amplification of competent CD34+CD38-cells. Exp Hematol 28 (7), 841-852; and Cibelli, J. B. et al. (2002) Parthenogenetic stem cells in nonhuman primates. Science 295 (5556), 819 all of which references are incorporated by references in their entirety).

A key step in the methods of the invention is the mixture of pluripotent or multipotent stem cell with developing or developed allogeneic or xenogeneic cells, particularly a mixture of different cell allogeneic or xenogeneic cell types where the mixture of cells comprises cells from more than one embryonic germ layer. For instance, the pluripotent or multipotent cells of the invention may be mixed with animal teratoma or teratocarcinoma cells in order to generate chimeric structures. Alternatively early mammalian embryos or fetal organs or organ systems could be dissociated or minced and mixed with the pluripotent or multipotent cells in vitro or in vivo. The methods of the invention will also help identify specific stages of embryonic development or organogenesis that provide the most appropriate environment or cell mixtures conducive for the development of specific cell types. Cells could also be included in the chimeric structure or mixture that secrete or release various molecules or growth factors that encourage development along a certain lineage and/or discourage development along other lineages.

In a preferred embodiment endothelial cells or stromal cells; or constituents thereof, e.g. membranes, soluble factors such as proteins and/or DNAs thereof will be used to promote differentiation. Such endothelial cells or stromal cell inducers will ideally be derived from the tissue or organ of a lineage that the embryonic cell is induced or promoted to differentiate into. For example, suitable endothelial cells may be derived from the kidney, liver, brain, heart, intestine, pancreas, stomach, eye, ear, bone, skin, et al.

Stromal cell suitable for use in the invention methods include those derived from the kidney, liver (to induce differentiation of hepatocytes and hematopoietic stem cells), brain, hear, intestine, pancreas, stomach, eye, ear, bone, skin, et al.

Such stromal and endothelial cells can be used in combination to produce desired tissues and may be fetal, adult or embryonic. Additionally, the membranes or soluble factors may be derived from such cells or may be produced by recombinant methods and used to promote differentiation.

Optionally, other signals, proteins, hormones, cytokines or factors as found in the appropriate environment could also be included in the mixture. Examples thereof include basic fibroblast growth factor, transforming growth factor, platelet derived growth factor, vascular endothelial growth factor, epidermal growth factor, epidermal growth factor, insulin-like growth factor, leukemia inhibitory factor, HGF, steel factor, VEGF, hepatocyte growth factor, insulin, erythropoietin, and colony stimulating growth factor CSF, GM-CSF, CCFF, etc. Examples of suitable hormone additions include estrogen, progesterone, and glucocorticoids, such as dexamethasone. Examples of cytokine additions include interferons, interleukins, and tumor necrosis factors (alpha or beta) among others. A list of potential suitable hormones, growth factor and cytokines and other culture constituents is set forth below:

Examples of growth factors, chemokines, and cytokines that may be tested in the disclosed assays include but are not limited to the Fibroblast Growth Factor family of proteins (FGF1-23) including but not limited to FGF basic (146 aa), FGF basic (157 AA), FGF acidic, the TGF beta family of proteins including but not limited to TGF-beta 1, TGF-beta 2, TGF-beta sRII, Latent TGF-beta, the Tumor necrosis factor (TNF) superfamily (TNFSF) including but not limited to TNFSF1-18, including TNF-alpha, TNF-beta, the insulin-like growth factor family including but not limited to IGF-1 and their binding proteins including but not limited to IGFBP-1, II-1 R rp2, IGFBP-5, IGFBP-6, the matrix metalloproteinases including but not limited to MMP-1, CF, MMP-2, CF, MMP-2 (NSA-expressed), CF, MMP-7, MMp-8, MMP-10, MMP-9, TIMP-1, CF, TIMP-2and other growth factors and cytokines including but not limited to PDGF, Flt-3 ligand, As Ligand, B7-1(CD80), B7-2(CD86), DR6, IL-13 R alpha, IL-15 R alpha, GRO beta/CXCL2 (aa 39-107), IL 1-18, II-8/CXCL8, GDNF, G-CSF, GM-CSF, M-GSF, PDGF-BB, PDGF-AA, PDGF-AB, IL-2 sR alpha, IL-2 sR beta, Soluble TNF RII, IL-6 sR, Soluble gp130, PD-ECGF, IL-4 sR, beta-ECGF, TGF-alpha, TGF-beta sRII, TGF-beta 5, LAP (TGF-beta 1), BDNF, LIF sR alpha, LIF, KGF/FGF-7, Pleiotrophin, ENA-78/CXCL5, SCF, beta-NGF, CNTF, Midkine, HB-EGF, SLPI, Betacellulin, Amphiregulin, PIGF, Angiogenin, IP-10ICXCL10, NT-3, NT-4, MIP-1 alpha/CCL3, MIP-1 beta/CCL4, I-309/CCL1, GRO alpha/CXCL1, GRO beta/CXCL2, GRO gamma/CXCL3, Rantes/CCL5, MCP-1/CCL2, MCP-2/CCL8, MCP-3/CCL7, IFN-gamma, Erythropoietin, Thrombopoietin, MIF, IGF-I, IGF-II, VEGF, HGF, Oncostatin M, HRG-alpha (EGF Domain), TGF-beta 2, CNTF R alpha, Tie-2/Fc Chimera, BMP-4, BMPR-IA, Eotaxin/CCL11, VEGF R1 (Fit-1), PDGF sR alpha, HCC-1/CCL 14, CTLA-4, MCP-4/CCL13, GCP-2/CXCL6, TECK/CCL25, MDC/CCL22, Activin A, Eotaxin-2/MPIF-2/CCL24, Eotaxin-3/CCL-26 (aa 24-94), TRAIL R1 (DR4), VEGF R3 (Fit-4)/SDF-1 alpha(PBSF)/CXCL12, MSP, BMP-2, HVEM/VEGF R2 (KDR), Ephrin-A3, MIP-3 alpha/CCL20, MIP-3 beta/CCL19, Fractalkine/CX3CL1 (Chemokine Domain), TARC/CCL17, 6Ckine/CCL21, p75 Neurotrophin R (NGF R), SMDF, Neurturin, Leptin R/Fc Chimera, MIG/CXCL9, NAP-2/CXCL7, PARC/CCL18, Cardiotrophin-1 (CT-1), GFR alpha-2, BMP-5, IL-8/CXCL8 (Endothelial Cell Derived), Tie-1, Viral CMV UL146, VEGF-D, Angiopoietin-2, Inhibin A, TRANCE/RANK L, CD6/Fc Chimera, CF, dMIP-1 delta/LKN-1/CCL15(68 aa), TRAIL R3/Fc Chimera, Soluble TNF RI, Activin RIA, EphA1, E-Cadherin, ENA-70, ENA-74, Eotaxin-3/CCL26, ALCAM, FGFR1 alpha (IIIc), Activin B, FGFT1 beta (IIIc), LIGHT, FGFR2 beta(IIIb), DNAM-1, Follistatin, GFR alpha-3, gp 130, I-TAC/CXCL11, IFN-gamma RI, IGFBP-2, IGFBP-3, Inhibin B, Prolactin CF, RANK, FGFR2 beta (IIIc), FGFR4, TrkB, GITR, MSP R, GITR Ligand, Lymphotactin/XCL1, FGFR2 alpha (IIIc), Activin AB, ICAM-3 (CD50), ICAM-1 (CD54), TNF RII, L-Selectin (CD62L, BLC/BCA-1/CXCL13, HCC-4/CCL16, ICAM-2 (CD102), IGFBP-4, Osteoprotegerin)OPG), uPAR, Activin RIB, VCAM-1 (CD106), CF, BMPR-II, IL-18 R, IL-12 R beta 1, Dtk, LBP,, SDF-I alpha (PBSF)/CXCL12 (synthetic), E-Selectin (CD62E), L-Selectin (CD62L), P-Selectin (CD62P), ICAM-1 (CD54), VCAM-1 (CD106), CD31 (PECAM-1),hedgehog family of proteins, Interleukin-10, Epidermal Growth Factor, Heregulin, HER4, Heparin Binding Epidermal Growth Factor, bFGF, MIP-18, MIP-2, MCP-1, MCP-5, NGF, NGF-B, leptin, Interferon A, Interferon A/D, Interferon B, Interferon Inducible Protein-10, Insulin Like Growth Factor-II, IGBFBP/IGF-1 Complex, C10, Cytokine Induced Neutrophil Chemoattractant 2, Cytokine Induced Neutrophil Chemoattractant 2B, Cytokine Induced Neutrophil Chemoattractant 1, Cytokine Responsive Gene-2, and any fragment thereof and their neutralizing antibodies.

Factors involved in cell-cell interactions that may be tested include but are not limited to the ADAM (A Disintegrin and Metalloproteinase) family of proteins including ADAM 1,2,3A, 3B, 4-31 and TS1-9, ADAMTSs (ADAMs with thrombospondin motifs), Reprolysins, metzincins, zincins, and zinc metalloproteinases and their neutralizing antibodies.

Adhesion molecules that may be tested include but are not limited to Ig superfamily CAM's, Integrins, Cadherins and Selectins and their neutralizing antibodies.

Nucleic acids that may be tested include but are not limited to those that encode or block by antisense, ribozyme activity, or RNA interference transcription factors that are involved in regulating gene expression during differentiation, genes for growth factors, cytokines, and extracellular matrix components, or other molecular activities that regulate differentiation.

Extracellular matrix components that may be tested include but are not limited to Keratin Sulphate Proteoglycan, Laminin, Chondroitin Sulphate A, SPARC, beta amyloid precursor protein, beta amyloid, presenilin 1,2, apolipoprotein E, thrombospondin-1,2, Heparan Sulphate, Heparan sulphate proteoglycan, Matrigel, Aggregan, Biglycan, Poly-L-Ornithine, the collagen family including but not limited to Collagen I-IV, Poly-D-Lysine, Ecistatin (Viper Venom), Flavoridin (Viper Venom), Kistrin (Viper Venom), Vitronectin, Superfibronectin, Fibronectin Adhesion-Promoting peptide, Fibronectin Fragment III-C, Fibronectin Fragment-30 KDA, Fibronectin-Like Polymer, Fibronectin Fragment 45 KDA, Fibronectin Fragment 70 KDA, Asialoganglioside-GM, Disialoganglioside-GOLA, Monosialo Ganglioside-GM₁, Monosialoganglioside-GM₂, Monosialoganglioside-GM_{3,}, Methylcellulose, Keratin Sulphate Proteoglycam, Laminin and Chondroitin Sulphate A.

Media components that may be tested include but are not limited to glucose concentration, lipids, transferrin, B-Cyclodextrin, Prostaglandin F₂, Somatostatin Thyrotropin Releasing Hormone, L-Thyroxine, 3,3,5-Triiodo-L-Thyronine, L-Ascorbic Acid, Fetuin, Heparin, 2-Mercaptoethanol, Horse Serum, DMSO, Chicken Serum, Goat Serum, Rabbit Serum, Human Serum, Pituitary Extract, Stromal Cell Factor, Conditioned Medium, Hybridoma Medium, d-Aldosterone, Dexamethasone, DHT, B-Estradiol, Glucagon, Insulin, Progesterone, Prostaglandin-D₂, Prostaglandin-E₁, Prostaglandin-E₂, Prostaglandin-F₂, Serum-Free Medium, Endothelial Cell Growth Supplement, Gene Therapy Medium, MDBK-GM Medium, QBSF-S1, Endothelial Medium, Keratinocyte Medium, Melanocyte Medium, Gly-His-Lys, soluble factors that inhibit or interfere with intracellular enzymes or other molecules including but not limited to compounds that alter chromatin modifying enzymes such as histone deacetylases such as butyrate or trichostatin A, compounds that modulates cAMP, protein kinanse inhibitors, compounds that alter intracellular calcium concentration, compounds that modulate phosphatidylinositol.

Environmental conditions that may be tested include but are not limited to oxygen tension, carbon dioxide tension, nitric oxide tension, temperature, pH, mechanical stress, altered culture substrates such as two vs. three dimensional substrates, growth on beads, inside cylinders, or porous substrates.

The particular hormones, growth factors and cytokines and culture conditions will vary depending upon the particular cell type that is to be provided.

The cell and tissue mixtures made according to the invention can also be used to screen for fetal and embryonic environmental proteins, hormones and other factors that contribute to cell development and differentiation, i.e., by exposing a mixture of the invention to various proteins, hormones and factors to determine which encourage or inhibit cells to develop along a certain developmental path. The proteins, hormones and factors thereby identified would also be included in the present invention.

Following mixture of the cells or inclusion of pluripotent or multipotent cells in allogeneic or xenogeneic embryonic structures, the cells are implanted or injected into an animal, fetus or embryo or cultured in vitro for further development. Prior to introducing the mixture of cells into the environment of a host animal, or culturing in vitro the mixture of cells may be aggregated with a biocompatible carrier material prior to being implanted into said suitable host embryo, fetus or animal. Such carrier materials are known in the art and include proteins such as collagen, gelatin, fibrin/fibrin clots, demineralized bone matrix (DBM), Matrigel® and Collastat®; carbohydrates such as starch, polysaccharides, saccharides, amylopectin, Hetastarch, alginate, methylcellulose and carboxymethylcellulose; proteoglycans, such as hyaluronate; agar; synthetic polymers, including polyesters, especially of normal metabolites such as glycolic acid, lactic acid, caprolactone, maleic acid, and glycols, polyethylene glycol, polyhydroxyethylmethacrylate, polymethylmethacrylate, polyamino acids, polydioxanone, and polyanhydrides; ceramics, such as tricalcium phosphate, hydroxyapatite, alumina, zirconia, bone mineral and gypsum; glasses such as Bioglass, A-W glass, and calcium phosphate glasses; metals including titanium, Ti-6Al-4V, cobalt-chromium alloys, stainless steel and tantalum; and hydrogel matrices.

As used in the present invention, the term “structure” is used to denote a mixture of cells that is more solid than fluid. For instance, a teratoma would be defined as a structure, as would a cohesive conglomeration of different cell or tissue types. “Structure” would also encompass a mixture of different cells that had been annealed together by way of Matrigel or some other suitable carrier such as those listed above. For the purposes of the present invention, a teratoma is defined as a group of differentiated cells containing one or more derivatives of mesoderm, endoderm, or ectoderm cells.

Suitable host embryos, fetuses or animals for further encouraging differentiation of the desired cells may be any animal, but preferred animals include mice, rats, guinea pigs, hamsters, non-human primates (cynomologus monkey and chimpanzee, for instance), sheep, pigs, cows. Typically, a suitable host fetus or animal is immuno-compromised, such as a SCID or nude mouse, or immuno-suppressed, i.e., with the aid of immunosuppressant drugs, or tolerized. For instance, a host fetus or animal may be tolerized by exposure to antigens, cells or tissues prior to the development of self-recognition. As an example, a developing sheep does not begin to develop self-recognition until the age of 60 days (continuing to about 85 days), so it is possible to introduce human cells before about day 55 to 60 and have the animal be tolerized to human cells that are implanted at a later time. Thereafter, the human cells may be differentiate without adverse immune response, even until the end of term, i.e., 145 days for sheep. Such a strategy is particular useful for implanting cells into organs or organ environments that are not suitably formed until after the development of self recognition, i.e., the thymic environment.

When implanted into a fetus or an adult animal, the mixtures or structures of the invention may be implanted or injected into any suitable organ or location, for instance, into the thymus, lungs, muscle wall, liver, heart, brain, pancreas, kidney, under the kidney capsule, into the peritoneum, etc. of said host fetus or animal. The present invention provides an advantage over methods of the prior art in that the mixtures and structures of cells provide a preliminary environment of cellular signaling for encouraging the development of cells, and the in vivo implantation serves to further that interaction. Thus, it will be possible to obtain a wider variety of differentiated cells from pluripotent and multipotent precursors than would be obtained by implanting single cells into fully formed organs.

The mixtures and structures may also be implanted or injected into a suitable host embryo. The mixture of cells may be implanted or injected into the endoderm, mesoderm or ectoderm of said suitable host embryo, or into overlapping or interconnecting regions, or into specific regions derived therefrom. When the mixture of cells is implanted or injected into the ectoderm of the host embryo, it may be implanted or injected into the general body ectoderm, the neural plate, the neural crest or the ectodermal placodes of said ectoderm, or into specific regions derived therefrom. When the mixture of cells is implanted or injected into the mesoderm of the host embryo, it may be implanted or injected into the paraxial mesoderm, the intermediate mesoderm or the lateral plate, or into specific regions derived therefrom. The mixture of cells may also be implanted or injected following segmentation of the paraxial mesoderm into a mesodermal somite, or following division of the lateral plate mesoderm into the intraembryonic splanchnopleure, or into specific regions derived therefrom.

The present invention also includes methods of obtaining the differentiated cells produced according to the invention. For instance, the invention includes a method of obtaining differentiated mammalian cells or tissues, comprising:

(a) obtaining a pluripotent or multipotent stem cell;

(b) mixing said pluripotent or multipotent stem cell with developing or developed allogeneic or xenogeneic cells;

(c) implanting or injecting said mixture of cells into a suitable host embryo, fetus or animal so as to generate differentiated mammalian cells or tissues; and

(d) obtaining said differentiated mammalian cells or tissues from said suitable host embryo, fetus or animal.

The present invention further includes methods of producing differentiated mammalian cells or tissues, e.g. human cells or tissues including the following steps:

(a) obtaining a pluripotent or multipotent cell;

(b) mixing said pluripotent or multipotent cell with developing allogeneic or xenogeneic cells;

(c) co-culturing said mixture in vitro, under conditions that the pluripotent or multipotent cells give rise to a desired differentiated cell or tissue types;

(d) obtaining said desired differentiated cell or tissue from the culture.

Such culturing may be effected on tissue culture plates or dishes, in apparatus that mimic in vivo conditions, in suspension cultures, etc. in the presence or absence of feeder layers using appropriate growth factors, hormones, cytokines, salts for differentiation. In some instances it may be desirable to include biocompatible polymeric matrices that promote cells to differentiate into tissues having the appropriate morphology and vascularization as the corresponding native tissue type.

Cells can be isolated using any means known in the art. For instance, pluripotent cells can be transfected with a heterologous DNA construct encoding a selectable marker prior to differentiation that can later be used to isolated the cells from surrounding cells and tissues by applying selection. For instance, such selectable markers include aminoglycoside phosphotransferase, puromycin, zeomycin, hygromycin, GLUT-2 and non-antibiotic resistance markers such as those described in U.S. Pat. No. 6,162,433, herein incorporated by reference. Selection may also be commenced during in vivo development such that the developing pluripotent cells survive while the other cells in the chimeric structures are selected against. Such in vivo selection may be commenced, for instance, after the chimeric structure has served the purpose of encouraging cells along a particular path, and the next level of encouragement is to be gleaned from the surrounding in vivo environment.

Alternatively, differentiated cells or tissues may be isolated using immunoaffinity purification or, in the case of differentiated cells, FACS. Immunoaffinity purification can be targeted to any cell surface molecule, whether it be one that is generally expressed on the surface of the desired cells, i.e., a native molecule, or whether it be a cell surface molecule, protein, or fusion protein expressed from a heterologous DNA construct transfected into the cells with the intent to use the molecule as a means for effecting purification. Any cell surface molecule can be used so long as it sufficiently distinguishes the desired cells from the surrounding cells such that purification may be effected.

The present invention also encompasses the differentiated cells or tissues produced by the methods described herein, as well as the chimeric mixtures and structures made to facilitate the differentiation of pluripotent and multipotent cells. Also included are methods of using the differentiated cells, tissues and organs for treating a patient in need of replacement cells or tissues, by transplanting into said patient the cells or tissues produced by the methods described herein, e.g., for the treatment of burns, blood disorders, cancer, chronic pain, diabetes, dwarfism, epilepsy, heart disease such as myocardial infarction, hemophilic, infertility, kidney disease, liver disease, osteoarthritis, osteoporosis, stroke, affective disorders, Alzheimer's disease, enzymatic defects, Huntington's disease, hypocholesterolemine, hypoparathyroidase, immunodeficiencies, Lou Gehrig's disease, macular degeneration, multiple sclerosis, muscular dystrophy, Parkinson's disease, rheumatoid arthritis, and spinal cord injuries. It may also be possible to transplant the chimeric mixtures and structures of cells into a patient in need of said replacement cells in order to achieve the desired cells via in vivo, in-patient development.

The present invention further contemplates the introduction of differentiated cells and tissues produced according to the disclosed methods into vascularized partial microtissue/microorganism arrays seen as disclosed in U.S. Pat. No. 6,197,575, incorporated by reference in its entirety herein, and the use thereof for high throughput screening, e.g. against potential therapeutic agents.

In order to further describe and illustrate the invention the following examples are provided.

### EXAMPLE 1

This experiment is designed to test the developmental potential of chimeric cell and tissue mixtures in an immunocomprised animal. This example is relevant to the methods whereby pluripotent stem cells may be mixed with allogeneic or xenogeneic cells or tissues, and implanted or injected into a SCID mouse or other immunocomprised animal in order to generate differentiated cells and tissues, e.g., for transplantation or replacement tissue.

First, the development of ES cells and ICM cells alone without being mixed were tested for teratoma formation following injection in the hind leg of SCID mice. ES cells tranfected with GFP were derived from two adult Holstein steers (two different ES cell lines were derived from each animal). ICMs were derived from twelve-day-old blastocysts. No more than about 100 cells each, in no more than 200 microliters each, were loaded into a 1-ml syringe. ICMs were mechanically isolated and loaded into a 1 ml syringe in a volume of 100 to 150 microliters. Twenty-two gauge needles were used for injection.

Bovine stem cells and ICMs that were injected into the skeletal muscle of SCID mice were retrieved after 7-8 weeks (although it is possible to permit cells to go longer or to remove them sooner). Small modular lesions were observed in two of the mice that received ES cell injections (mice#s 7 & 9).

Gross Examination:

A 2×2 mm-sized milky white nodule was retrieved from the right hind leg near the sciatic nerve of mouse #7. This corresponds with the injection of three plates of ES 22.C. A 1×1 mm sized milky white nodule was identified within the muscle tissue of mouse #9, which corresponds to the injection of three plates of ES 25.F.

Histologic Analysis:

Mouse#7: Histologic sections of the teratoma were analyzed with hematoxylin and cosin (H&E), safranin-O and immunocytochemistry using cytokertin (AE1/AE3) and alpha smooth muscle actin antibodies.

H&E: The injected cells formed a round tissue mass within the skeletal muscle tissue. The teratoma consisted of four different sized compartments with the cellular debris in the center. Tissue formation was noted on the wall of each compartment (data not shown). Epithelial (round nuclei) and stromal cells (spindle-shaped nuclei) were observed in the teratoma tissue (data not shown). There was no evidence of cartilage, bone or adipose tissue.

Safranin O: Negative staining was obtained, which indicates the absence of cartilage tissue formation.

Immunocytochemistry with AE1/AE3 antibodies: The teratoma section showed positively stained epithelial cells (data not shown).

Immunocytochemistry with alpha smooth actin antibodies: Small islands of positively stained muscle tissue was observed within the teratoma (data not shown). The retrieved tissue demonstrated epithelial, smooth muscle, and stromal tissue compartments. Cartilage, bone and adipose tissue were not identified in the teratoma.

Mouse# 9: Histologic analysis on the retrieved nodule demonstrated a skeletal muscle mass. Microscopic analysis demonstrated that no other tissue formed.

Thus, bovine ES cells and ICM cells injected into the hind leg of SCID mice respond to environmental cues and differentiate into epithelial, muscle and stromal tissue derivatives. Next, cells will be tested for developmental potential following injection into other sites in the SCID mice, alone and following mixture with different chimeric combinations of isogenic, allogeneic and xenogeneic cells and tissues.

### EXAMPLE 2

This experiment is designed to test the developmental potential of chimeric cell and tissue mixtures in a tolerized animal (sheep).

A developing sheep does not begin to develop self-recognition until the age of 60 days (continuing to about 85 days), so it is possible to introduce human cells before about day 55 to 60 and have the animal be tolerized to human cells that are implanted at a later time. Thereafter, the human cells may be differentiate without adverse immune response, even until the end of term, i.e., 145 days for sheep. Such a strategy is particular useful for implanting c ells into organs or organ environments that are not suitably formed until after the development of self recognition, i.e., the thymic environment.

To demonstrate this utility, different combinations of chimeric allogeneic and xenogeneic cell and tissues mixtures will be implanted or injected into different sites in an intrauterine sheep fetus at different times during development, and particularly before the development of self recognition at day 55-65. The cell mixture implants will be examined at different times and also after full development to determine what types of differentiated cells result from the various mixtures, and at different locations including the umbilical cord. Variations in development according to the time and place of implantation will be documented.

Using standard sterile surgical techniques, the maternal abdomen will be opened in the midline, taking care to avoid the large ventral vein. The uterus will be exposed and both horns evaluated to determine the number of fetuses. The uterine horn will then be wrapped in wet warm towels, and the uterus incised along the avascular plane using electrocautery. The fetus will then be exposed, taking care to avoid entanglement or kinking of the umbilical cord. The amniotic fluid is partially removed and kept in a sterile reservoir, at 37° C. The fetus will then undergo surgical implantations of tissue engineered constructs containing non-human primate's primitive stem cells and/or injections of those cells, in free suspension, at several different anatomic sites. If a fetal laparotomy or thoracotomy is performed, its closure will be in layers, through standard technique. When the fetal operation is complete, the fetus is returned to the uterus. The amniotic fluid is then reinfused and/or partially replaced with isothermic Lactate Ringer's solution, until the uterus is full. Antibiotics are then injected into the amniotic fluid (Cefazolin-500 mg per horn), and the uterus closed using a titanium TA-90 stapler. The maternal abdominal wall is then closed in layers. Induction and maintenance of anesthesia will be accomplished with inhaled isoflurane or halothane (2-3% in 60-100% oxygen).

In some cases, animals will be euthanized for early analysis. In others, normal delivery will be allowed. No impairment is expected. However, should any unforeseen complication of stem cell differentiation ensue and lead to any discomfort to the animals that could not be treated, euthanasia will be performed immediately. Pain should only be present in the immediate postoperative period and will be treated with analgesics, i.e., Buprenorphine, 0.01-0.02 mg/Kg IM.

### EXAMPLE 3

In another experiment multiple injections of parthenogenically derived Cyno-1 stem cells (obtained by in vitro parthenogenic activation of an unfertilized Cyno oocyte) were made in the left atrium and the left ventricle of an approximately 3-month old sheep fetus. In this experiment a total of 0.55CC were used for the cell suspension (because of the way that Cyno-1 cells are harvested and grown on a feeder layer it was not feasible to make an exact cell count, however it is estimated that this suspension contained several million cells.

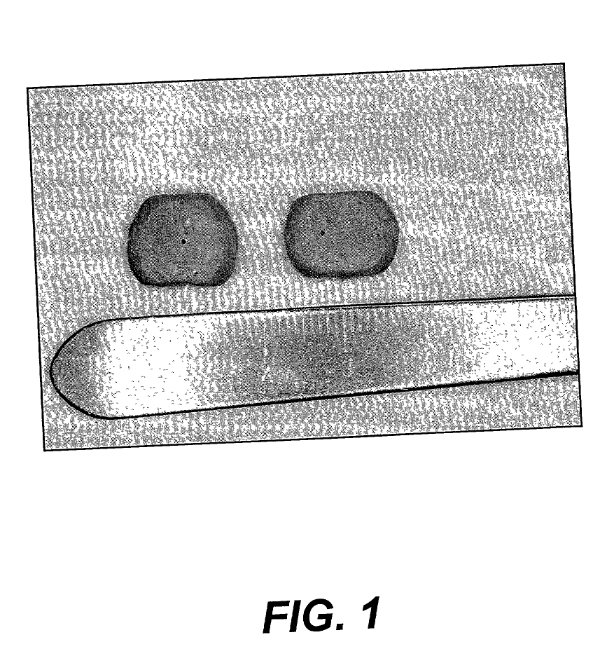

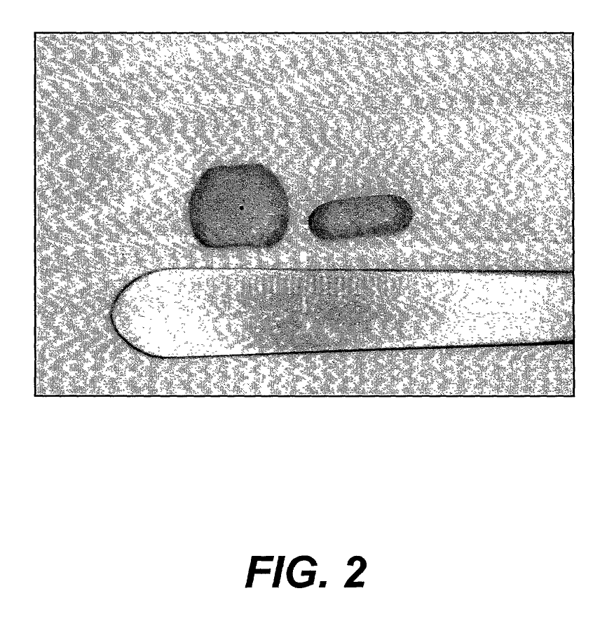

During this experiment the heart was beating and as a result some of the cell suspension escaped (oozed) into the surrounding thoracic cavity. It was discovered on surgical inspection of the thoracic cavity six weeks after injection of said primate stem cells that large discs of bone had formed and were free-floating in the thoracic cavity. (This can be seen in FIGS. 1 and 2). These results clearly establish that the thoracic-environment contains a cellular millieu that induces differentiation of the injected primate pluripotent cells, i.e., induced these stem cells to differentiate into bone cells. This experiment is ongoing. Additionally, experiments are ongoing to confirm that some of the primate stem cells which are injected into the heart became cardiac cells. This may be determined by PCR detection of donor stem cells in the heart of the treated sheep fetus.

### EXAMPLE 4

### Reconstitution of the Immune System of a Bovine by Nuclear Transfer

An experiment was conducted using cattle as an animal model for the treatment of autoimmune disease and other hematopoietic disorders in humans. The overall strategy is to replace endogenous bone marrow that is defective e.g., as a result of disease, genetic detect or age with cloned stem cells of the same donor. By using autologous cells to repopulate the patient's bone marrow, the need for donor matching and the risk of host vs. graft reactions are minimized or eliminated. Thus, the purpose of this experiment is to provide further proof that the bone marrow of a mature individual can be repopulated with autologous stem cells cloned using nuclear transfer techniques and that such cells will differentiate into appropriate cell types when exposed to developing or developed allogeneic or xenogeneic cells and the appropriate cellular millieu.

Because of the few number of stem cells that are produced by use of in vitro culture of cloned cells, cloned embryos are implanted into recipient donors and allowed to mature to 100 days of gestation. Thereafter, stem cells are harvested from the fetal liver. (Once it is established that bone marrow repopulation is feasible with cloned cells, in vitro culturing will be used to produce hematopoietic stem cells and other fetal stem cells from human ES cells). Additionally, the cow was selected as an animal model for these studies as cloning is well developed in cows, including embryo transfer of cloned cells that enable development of fetal stem cells for injection back into a recipient.

In this experiment one of the two cows that had a cloned embryo was given a drug to suppress bone marrow (as discussed in detail below). After 100 days of gestation the cloned fetuses were surgically removed from the recipient cows and three livers harvested. Fetal liver cells were then isolated and injected intravenously back into the cows from which they were originally cloned to reconstitute the bone marrow. The cow's peripheral blood and bone marrow were sampled periodically to monitor the progress of the autologous graft.

Materials and Methods Used for This Experiment

Two specific-pathogen-free non-lactating cows 10-13 years old were used for this study. Dermal skin biopsies were obtained from the ear of the animals for tissue culture, and were expanded for marker gene (PGK-Neo) transfection. Cells were selected with G418 for >10 days, and neomycin-resistant colonies isolated for nuclear transfer. Cloning of embryos was done at ACT as previously described (Cibelli et al, Science 280:1256-58(1998); Lanza et al, Science 294:1893-94 (2001)). The embryos are non-surgically implanted into recipient heifers at our Em Tran facility in Pennsylvania. At 100 days of gestation the fetuses were removed from the recipient cows by hysterectomy and flown by private jet to Dr. Malcolm Moore at the Sloane-Kettering Memorial Cancer Center where fetal liver cells were harvested by Ficoll separation and tested by PCR for presence of their transgenic (NeoR) marker. At this point, the two clone-donor cows had already been admitted to the New Bolton Large Animal Center at the University of Pennsylvania School of Veterniary Medicine. Myelosuppression was achieved in one of the animals by IV treatment with Busulfex (1 mg/kg lean weight per day for 4 days) with a drug washout peroid of 48 hours prior to infusion of the fetal liver cells. The fetal liver cell infusions were flown by private jet from Sloane Kettering to the New Bolton Center for IV administration to the original donor cows.

Each cow received the equivalent of one fetus-worth of ficoll-separated fetal liver cells (3-10×19e9 cells) suspended in 1 liter of sterile tissue culture media, infused over ½-1 hour. Post-treatment monitoring included daily physical examination; collection of blood (3 ml) for complete blood count daily for 14 days, then weekly for 3 months; collection of blood (5 ml) for chemistry screen weekly for one month, then monthly for 3 months; and collection of bone marrow (5 ml) by needle aspiration from the ileum following administration of a local anesthetic, monthly for 3 months. Larger volumes of blood were drawn prior to, 6 days, 12 days, 21 days, 60 days, and monthly thereafter, after the cell infusion for special testing such as flow cytometry and PCR testing for the NeoR marker added to the cloned cells to permit differentiation between native cells and transplanted cells.

### Isolation of Mononuclear Cells

Mononuclear cells are isolated from the blood for CFC assay and PCR of individual colonies. PCR is effected by TakMan of mononuclear cells and granulocytes, plus DNA obtained from granulocytes and mononuclear cells for telomere length experiments. The marrow is set up for CFC and CAFC/CTC-IC.

### Transplantation of Cells

Pre-transplant 500 ml blood draw is used as a baseline for responding lymphocytes and stimulating for in vitro proliferation and targets for cytolysine.

Aftertransplant 200 ml draws are taken (two time points within the 12 day recovery time, at 21 days and at monthly intervals thereafter.

### Cell Surface Phenotyping

Cell samples are analyzed for certain cell types on the basis of cell marker expression. Particularly, the following combination of markers are screened for:

(i) CD3, CD4, CD8, class I, class II, CD49E

(ii) CD25, CD45RO, and CD62L in double stain versus CD4 and CD8.

### Cell Function Assays

a) T cell proliferation

T cell proliferation is determined by use of phytonemagglutin assay (PHA)

(b) MLC

Mixed lymphocyte culture (MLC) is also effected to evaluate lymphocyte cell function. In these experiments two normal, allogeneic cows are used as stimulators and the pre-transplant bleed used as the synogeneic control.

(c) Complement Mediated Lysis (CML)

CML is evaluated using same allogeneic stimulators as above.

51 Cr-release is used to assay lytic function of cells.

(d) Elispot Assay

This assay is conducted to quantify proliferation or cytolysis.

(e) Natural Killer Cell Assay

Assays for NK actively are cells conducted.

### Results

Processed fetuses are analyzed. Fetus #404 appears intact, whereas other fetus #410 has an abdominal rupture in the region of the umbilicus with extrusion of intestines. (This is hypothesized to be a traumatic rupture that occurred while a specimen of umbilical cord blood was obtained).

The livers are removed from all fetuses, each liver weighed and a sample from each liver processed for DNA extraction and PCR studies. This DNA extraction is conducted a little more than an hour after harvesting of liver.

Cell suspensions are made from the liver simultaneous to DNA extraction, within several minutes to about 1 1.25 hours after liver removal. An autopsy is conducted on all fetuses and analysis made of the isolated spleen, stomach, large and small intestines, kidneys, heart, lung and trachea, uterus/ovaries, brain, eye, mediastinal tissue and thymus, and lower hind leg. All organs are photographed and tissue samples fixed in 10% formulating.

Cells are processed by ficoll centrifugation. A substantial pellet formed but interface contains significant number of cells. The cells are processed and about 1.3-3.5×10⁹ cells are obtained.

The cells are suspended in 50ml of 20% Fetal Calf Serum and phosphate buffered saline (PBS) in minibags. Each sample is placed in a styrofoam container with a bag of ice, and placed in a larger container for shipping with each bag identified by fetus identifier.

Fetal liver cells after transport are established in a methylcellulose culture and in a long-term MS5 stromal co-culture. Cytospins are effected for morphology. Cells are cryopreserved in DMSO and fetuses are frozen at −200° C.

As discussed above, the results of these experiments provide proof or principle as to the in vivo potential of hematopoietic stem cells to produce differentiated hematopoietic cell lineages in vivo, because these cells are exposed to the appropriate cellular millieu and cells that promote differentiation. Particularly, experiments were conducted wherein hematopoietic stem cells of obtained from the liver of a cloned bovine were transplanted into bovine animals and the effects of such transplantations studied over time.

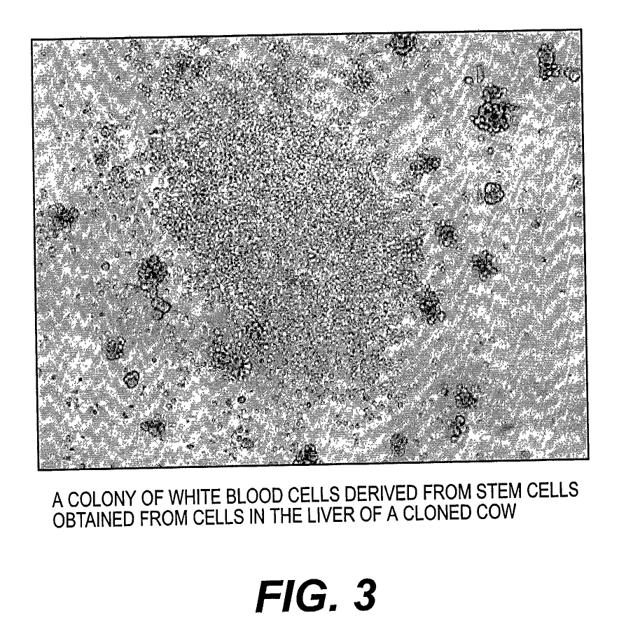

As shown in FIG. 3, and evaluation of cells obtained from blood samples drawn from the recipient animal, it can be seen that a colony of white blood cells resulted from transplantation of the transplanted HSCS.

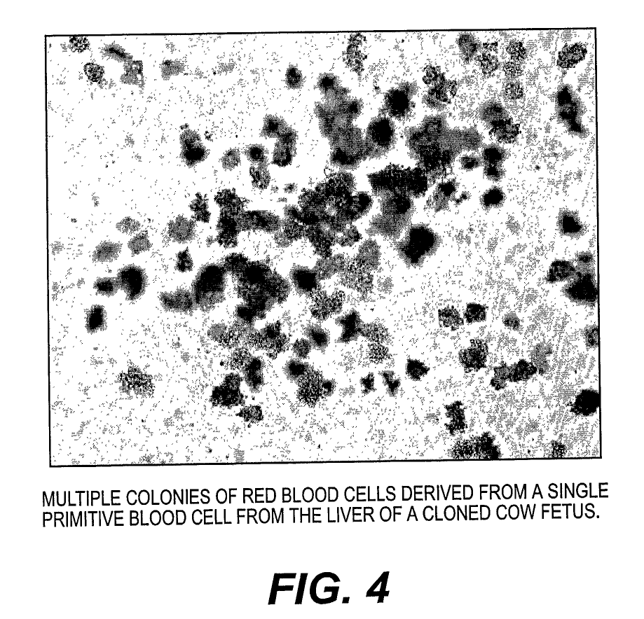

Also, as shown in FIG. 4, multiple colonies of red blood cells were produced in vivo from a single primitive blood cell derived from the liver of a cloned cow fetus.

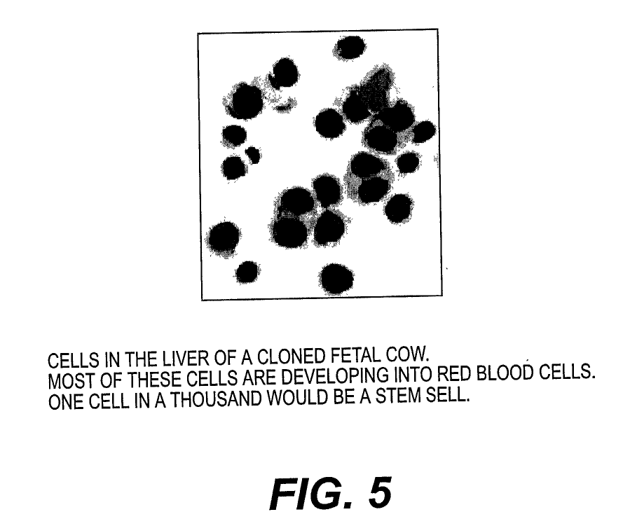

Additionally, FIG. 5 shows the presence of the transplanted cells in the liver of a cloned fetal cow. Upon inspection it is seen that most of these cells are developing into red blood cells. Of these cells, one cell in a thousand should be a stem cell.

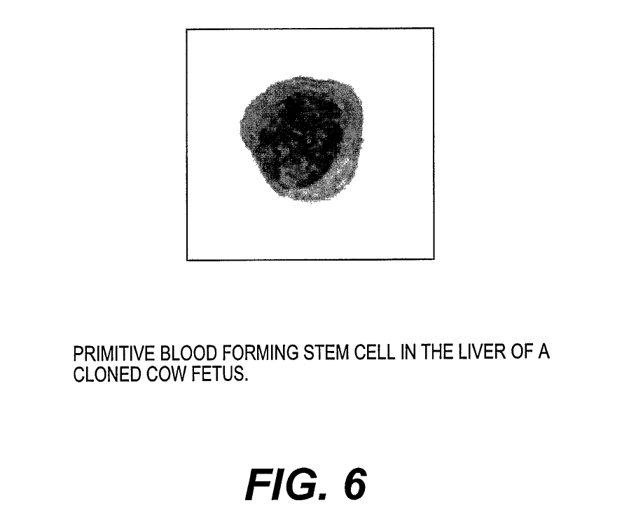

FIG. 6 shows a primitive blood forming stem cell (HSC) in the liver of a cloned cow fetus.

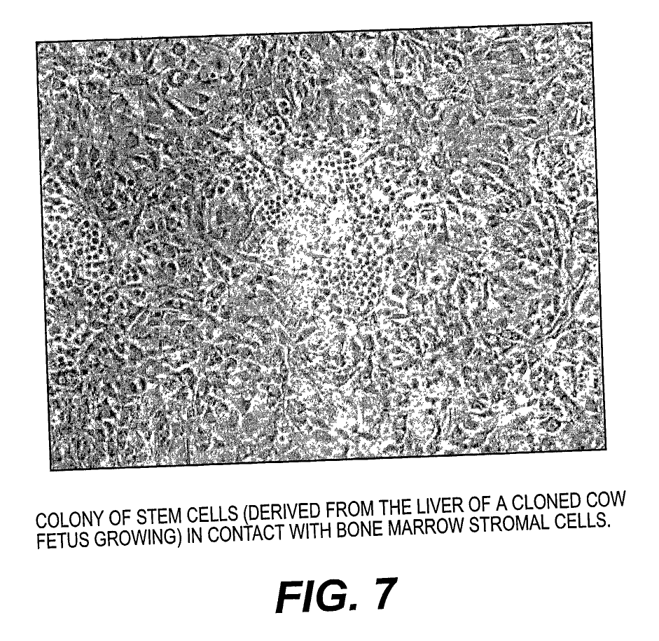

FIG. 7 shows a colony of stem cells derived from the liver of a cloned cow fetus growing in contact with bone marrow stromal cells.

These in vivo results are preliminary but provide convincing in vivo evidence that stromal cells derived from developing embryonic, fetal or adult tissues provide specific inductive signals that are important in the development of tissues and the regulation of growth and differentiation pathways. (As discussed elsewhere in this application, these results confirm that stromal or epithelial cells can be used in vitro or in vivo to induce or promote pluripotent stem cells to differentiate into specific pathways). Examples of types of stromal cells that may be used to promote specific differentiation pathways include those present in the brain, eye, pharyngeal pancreas, lungs, kidneys, liver, heart, intestine, pancreas, bone, cartilage, skeletal muscle, smooth muscle, ear, esophagus, stomach, blood vessels, Aorta-mesorephros (AGM) region t al.

The results contained in the Figures are especially compelling given that grafts of adult hematopoietic stem cells usually only repopulate a small percentage of the blood cells and also given that serial transplantation is ordinarily limited by replicative senescence and telomere shortening (See Brummendorf et al., Ann. NY Acad. Sci. 938:1-7 (2001)).

The very effective colonization and cell differentiation observed in this experiment may be partly a result of the use of cloned cells, which are believed more youthful and to posses lengthened telomeres relative to HSCs derived from adult animals or even relative to non-cloned fetuses.

The cells in the bone marrow which are believed to promote cloned fetal liver HSCs to differentiate into differentiate blood cell lineages are mesenchymal cells (Stro-It), perivascular lipocytes (desmint) and endothelial cells (CD34 +, FIK-1 +, Sca-1 +) (See Blazsek et al., Blood 96(12): 3763-71 (2000)).

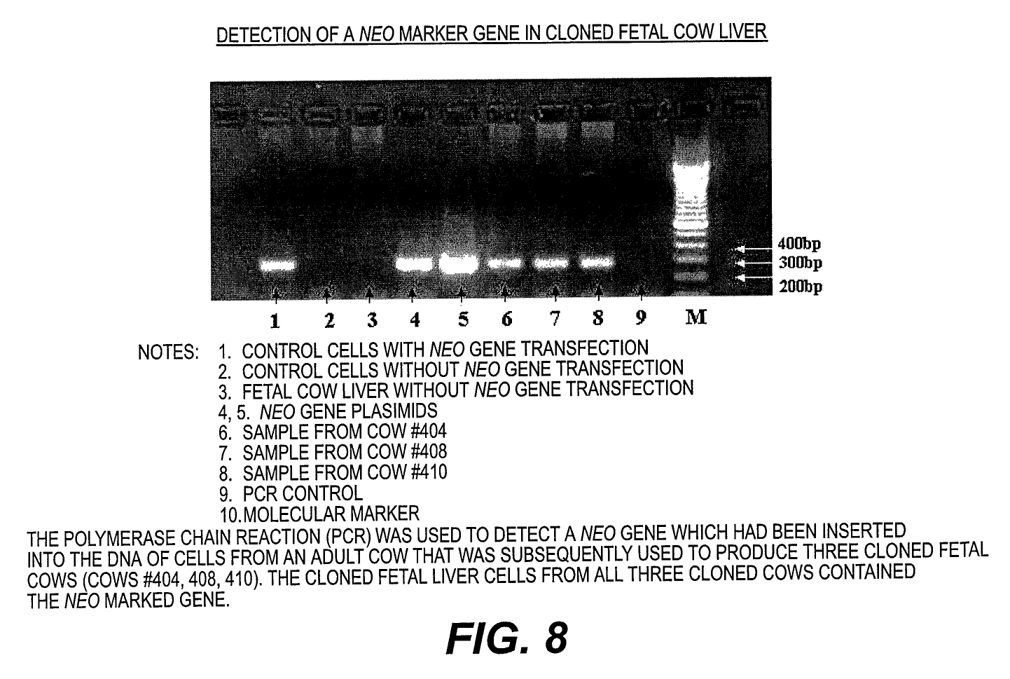

Polymerase chain reaction (PCR) was also used to detect the presence of the Neo gene which was inserted into the DNA of cells derived from an adult cow that was subsequently used to produce three cloned fetal cows (designed 404, 408, 410). As shown in FIG. 8 the cloned fetal liver cells from all three cloned cows contain the neo marker gene. Thus, the transplanted cells are detected in the cloned fetal cow liver.

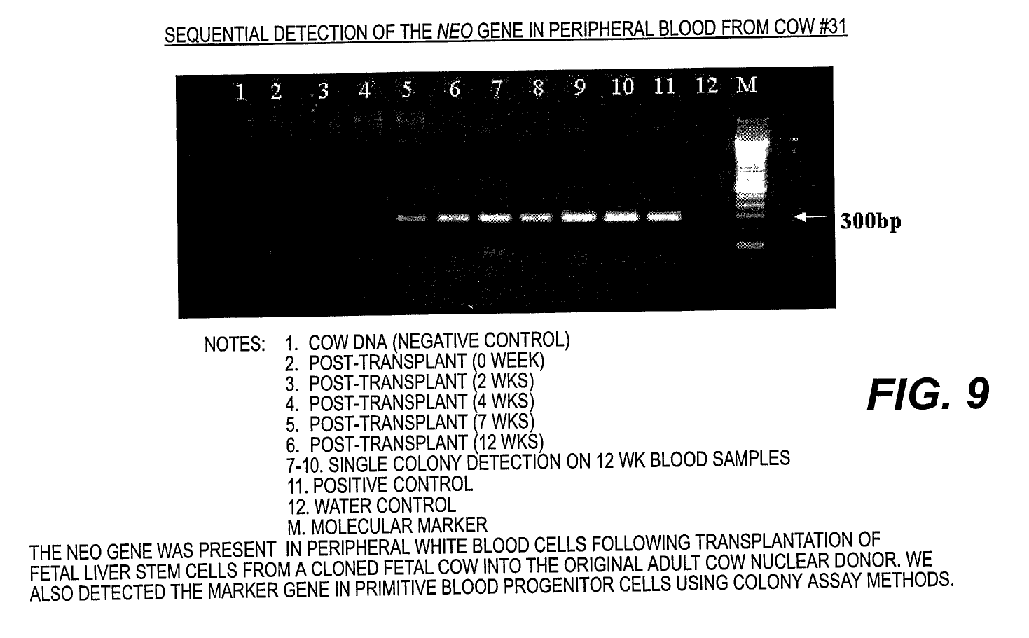

Additionally, PCR was conducted to detect the presence of the Neo gene in peripheral blood cells following transplantation of fetal liver stem cells from a cloned fetal cow into the original adult cow used for nuclear transfer. As shown in FIG. 9, the Neo gene is detected in peripheral while blood cells following transplantation. Also the neo marker is detected in primitive blood progenitor cells by colony assay methods.

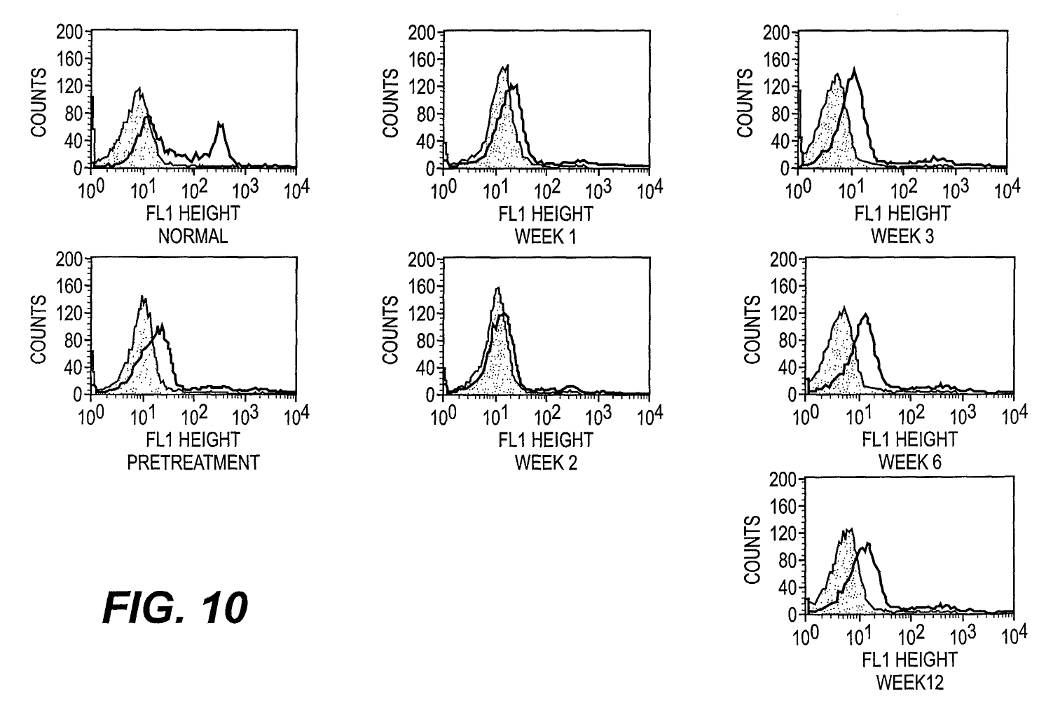

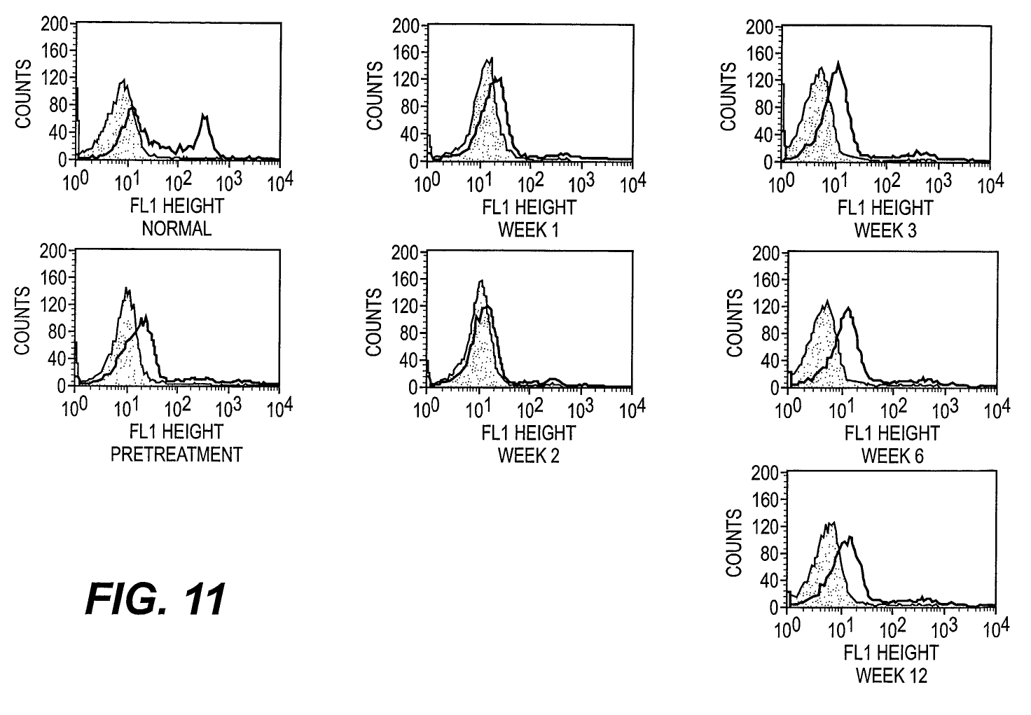

CFC assays are conducted as indicated above using mononuclear cells obtained from the blood. These assays are conducted using cells from an animal normal, and using cells from the transplant recipient pretreatment, week 1, week 2, week 6 and week 12 after transplantation of HSCS. These results are in FIGS. 10 and 11.

These results of these experiments as the data obtained to date suggests that one recipient (which did not receive any bone marrow inhibitory compound) had almost half of its immune system (˜40%) replaced with the donor (Neo R positive) cells. Functional studies of these cells are ongoing but this suggests that a minimal number of transplanted cells (˜a thimble full of cloned stem cells) could take over and repopulate the immune system of a 1500+ pound animal.

These results suggest that after the entire immune system of the recipient should be virtually replaced with that of the youthful, rejuvenated donor. This has significant therapeutic applications in the contexts of human therapy, e.g., the immune systems of human subjects that are immunocomprised as a result of disease, genetic defect or drug or radiotherapy may be replaced, potentially without the need for any myeloblative or suppressive drugs.

The results are further compelling based on the fact that grafts of HSCs usually repopulate a small percentage of the blood cells and several transplantation is limited by replicative Senescence and telomere shortening (Brummendorf et al., Ann NyAcad Sci 938:1-71 (2001)). These results suggest that the cells are more than normal, perhaps as a consequence of lengthened telomeres as a result of nuclear transfer.

### EXAMPLE 5

### In vitro Examples

Use of stromal cells to induce hematopoietic lineages in vitro. A co-culture of pluripotent stem cells such as human NT-derived ICMs on the macrophage colony-stimulating factor-deficient OP9 stromal cell line is effected. The ICM derived from an embryo is plated in juxtaposition with OP9 cells and incubated for 1-7 days and then serially passaged by mechanical enzymatic (e.g. trypsin) removal and then plated again on OP9 cells. The serial replating of these cells will differentiate the ICM into a mesenchymal stem cell that is CD34—but capable of causing long-term repopulation of the hematopoietic system. (The OP9 system was described previously by Nakano et al, 1994. Generation of lymphohematopoietic cells from embryonic stem cells in culture. Science, 265: 1098-1101.) The OP9 cells are a stromal cell line obtained from the calvaria of op/op mice. These have a mutation in the M-CSF gene. Since M-CSF inhibits hematopoiesis, these cells induce hematopoiesis with increased efficiency. Nakano et al described this method but not for ICMs or NT or parthenogentically-derived ES or ICMs. This has the advantage over the prior art and other published methods of co-culturing ES cells with yolk sac endothelial cells (Kaufman et al, 2001 Proc Natl Acad Sci USA, 98(19): 110716-10721) where it is unlikely that long-term repopulating cells are produced, and it is preferred over genetic modification technologies such the production of hematopoietic cells from the formation of embryoid bodies such as in methylcellulose in bacteria-grade Petri dishes where no long-term repopulating cells were achieved (Weiss & Orkin, 1996, In vitro differentiation of murine embryonic stem cells: new approaches to old problems, J Clin Invest. 97: 591-595).

See also Suzuki and Nakano, 2001, Int. J. Hematol. 73:1-5 which discloses a co-culture of OP9 and murine ES cells.

### i) Hematopoietic Cell Differentiation

Another example of a stromal cell line which may be used to induce hematopoiesis would be stromal cells from the Aorta-Gonad-Mesonephros (AGM) region. Such stromal cell cultures may be obtained from the intraembryonic AGM region of mice at 10.5-11.5 dpc or the equivalent stage of other human and nonhuman animal embryos. As described above, ES cells, ICMs and those obtained by nuclear transfer, parthenogenesis, or cytoplasmic transfer can be plated in juxtaposition with stromal cells and serially passaged to differentiated them into hematopoietic lineages. More preferably, the stromal cells may be co-cultured with endothelial cells from the AGM region purified as described below. The AGM endothelial cells express a podocalyxin-like protein (PCLP1) and PCLP1+CD45-endothelial cells are preferred.

### ii) Endothelium Example

Another example involves the use of endothelial cells to induce differentiation. Endothelial cells from various tissues show variations in morphology and molecular markers (Craig et al., Endothelial cells from diverse tissues exhibit differences in growth and morphology, Microvasc. Res 55(1) 65-76 (1998) though no one has reported the tissue specific induction of differentiation from pluripotent stem cells, such as ES or ICM cells.

Endothelial cells can be isolated from a wide array of tissues to induce the differentiation of pluripotent stem cells, such as ES cells, ICM cells, and so on as follows. A culture of the tissue-specific endothelial cells is obtained by techniques known in the art. For example, in the case of the AGM region, the tissue is minced under sterile and then incubated in isotonic saline with 0.2% interstitial collagenase until the tissue is desegregated. The endothelium cells are purified by affinity, flow, or other related techniques well known in the art. For example, the mixture of cells is mixed with magnetic beads coated with antibody directed to endothelial-specific surface antigens, including but not limited to antibody specific for E-selectin, PE-CAM/CD31, VEGF receptor, lectin ulex europaeus I (UEA-I), or other means to purify the endothelial cells from the mixture. In the case of AGM endothelial cells, the use of antibodies to PCLP1 are preferred (Hara et al, 1999, Identification of podocalyxin-like protein 1 as a novel cell surface marker for hemangioblasts in the murine aorta-gonad-mesonephros region, Immunity, 11: 567-578). An example of fluorescence-activated flow sorting would be the labeling of the endothelial cells with 10 micrograms/mL Dil-Ac-LDL for 4 h at 37 degrees C. then trypsinized and purifying the endothelial cells that take up the LDL by flow sorting.

Endothelial cells are then be plated in tissue culture conditions that favors the growth of endothelial cells, such as M199 medium supplemented with 10 ng/mL VEGF, 10U/mL heparin, 2-5 ng/mL bFGF, and 5-10% human serum.

A preferred example involves the use of intraembryonic AGM endothelial cells to induce hematopoietic stem cells, especially long-term repopulating hematopoietic stem cells. AGM endothelial stem cells are grown in culture, a nonlimiting example being the growth of the cells as a monolayer in a tissue culture dish. ES cells, ICM cells, etc. or downstream derivatives of these are then added to the tissue culture dish such that the two cells share the culture environment thereby allowing a cell-cell communication. For example, ES cells or ICMs can be grown directly on top of an irradiated endothelial layer for 5-30 days, preferably 18 days. The media contains 20% FBS but no other growth factors are added. At the end of this period of induction, the hematopoietic cells are aspirated, flow sorted using commonly used cell surface markers such as CD34. The use of endothelial cells from the developing AGM is preferred as no long-term repopulating cells should be obtained.

Another example involves those of endothelial cells to induce the differentiation of myocardial cells. Endothelial cells (e.g., those from the developing heart) are, placed in tissue culture and ES cells, ICM-derived cells or their derivatives are added to the tissue culture dish such that the two cells share the culture environment thereby allowing a cell-cell communication. For example, as a nonlimiting, ES cell can be grown directly on top of the endothelial layer.

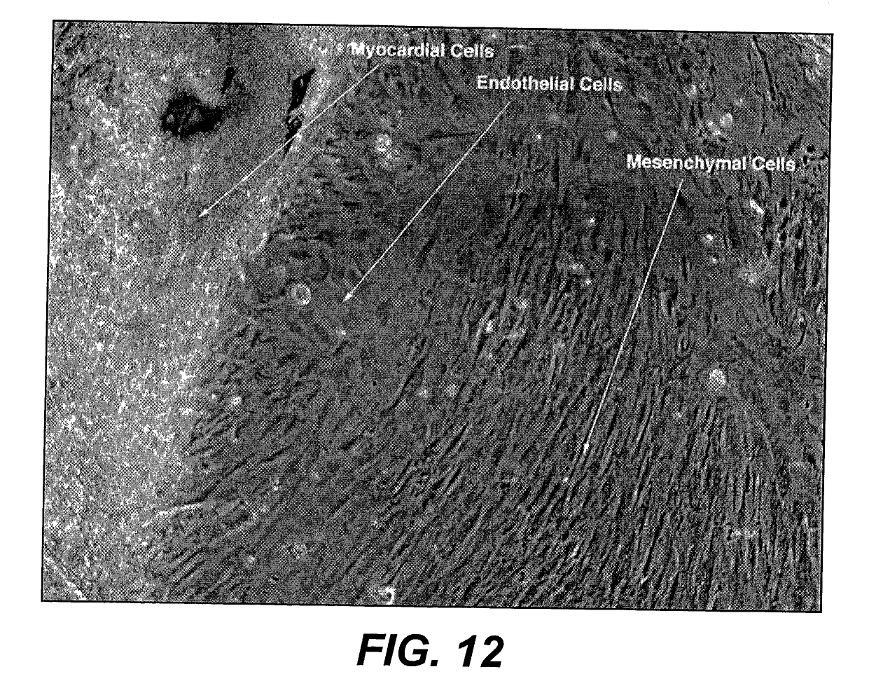

In fact the present assignee has obtained cells marked “endothelial cells” labeled with Di-Ac-LDL and they were positive which were obtained upon differentiate of a cynomologus ES cell line to produce a co-culture comprising mesenchymal cells, cardiac cells and endothelial cells (See FIG. 12). It can be seen that the endothelial and cardiac cells are juxtaposed providing in vitro evidence that these cells promote the development of pluripotent cells into cardiac cells. As shown in FIG. 12 beating cells near the cells with an endothelial cell morphology were observed.

Endothelial cells that induce myocardial differentiation can be isolated from spontaneous matches of myocardial development such as that shown above. Isolation is performed by labeling with DII-labeled LDL that is specifically taken up by vascular endothelial cells. The cells are removed from the culture dish, and flow sorted and the DII-labeled cells are replated as a relatively pure population of the endothelial cells. The endothelial cells that induce myocardial differentiation can then be propagated, cryopreserved, and used when convenient to induce myocardial differentiation in screening assays, or to produce myocardial cells for research or therapy.

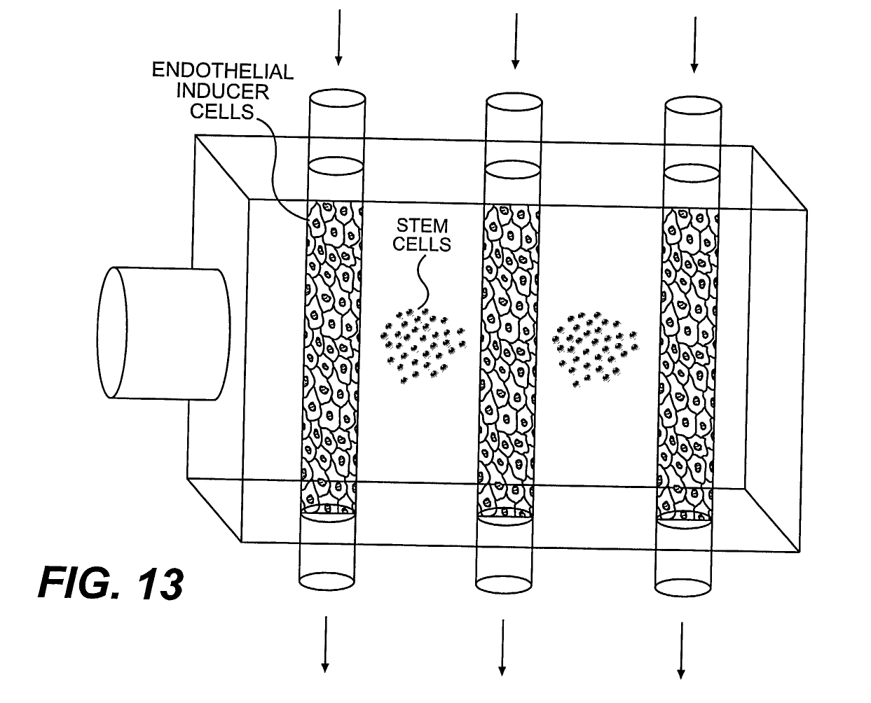

Three dimensional myocardial tissue (shown in FIG. 13 below) can be produced by providing induction in a three dimensional bioreactor. For example, endothelial cells that induce myocardial differentiation can be trypsinized and allowed to attach to polymer tubes that function as “molds” of blood vessels. The tubes allow media to perfuse and support endothelial attachment and viability. ES cells, ICM-derived cells, or other derivative cells are then cultured in the bioreactor to induce myocardial development. The artificial vessels are perfused with tissue culture media containing factors that support the growth of endothelium and myocardial differentiation. Factors which induce differentiation include those identified above, and preferably may comprise Brain-Derived Growth Factor (BDNF) and Vascular Endothelial Growth Factor-A (VEGF-A), in particular isoform 165.

Such a system can be used with many different endothelial cell types to generate cells and three-dimensional tissues. The endothelial cells can be embryonic, fetal, or adult in origin, and may be with or without genetic modification. The types of endothelial cells include, but are not limited to kidney, liver (to induce the differentiation of hepatocytes and hematopoietic stem cells), brain, heart, intestine, pancreas, stomach, eye, ear, bone, skin, and so on.

Thus, in one aspect the invention will involve the co-culture of inducing endothelium with the undifferentiated cells. The tissue culture vessel and its architecture may take other forms than that shown above to increase efficiency and to form tissues when growing tissues in two of three dimensions.

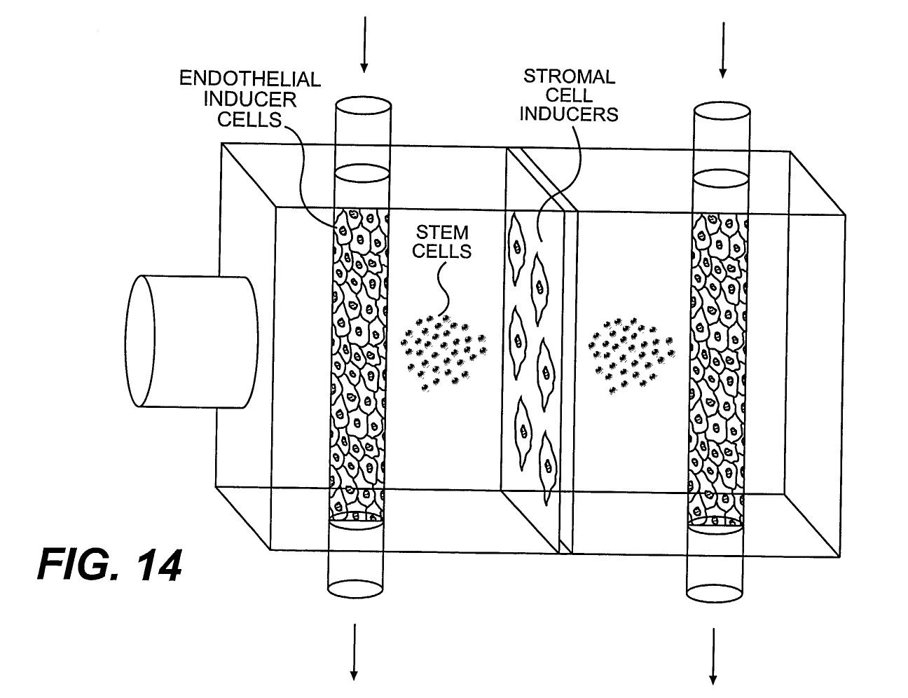

Another example is to combine endothelial cell inducers with stromal (for instance fibroblast) cell inducers. An example of how this can be effected is shown in FIG. 14.

Such a system can be used with many different endothelial and stromal cell types to generate cells and three-dimensional tissues. The endothelial and stromal cells can be of the same tissue of origin or of different tissues and may be embryonic, fetal, or adult in origin, and may be with or without genetic modification. The types of endothelial or stromal cells include, but are not limited to kidney, liver (to induce the differentiation of hepatocytes and hematopoietic stem cells), brain, heart, intestine, pancreas, stomach, eye, ear, bone, skin, and so on.

Thus, in another aspect the invention involves the co-culture of inducing endothelium and stromal cells with the undifferentiated cells. The vessel and its architecture may take other forms than that shown above to in crease efficiency and to form tissues when growing tissues in two of three dimensions.

### EXAMPLE 6

### Production of Pancreatic B-Cells

The production of pancreatic B-cells would be useful in the treatment of diabetes. These cells can be produced in a 3-step differentiation protocol as follows.

The first step is to direct the differentiation of B-cells. The pancreas normally forms from two an/agen, the ventral and dorsal pancreatic buds. The dorsal endoderm is in close proximity to the notochord and the ventral endoderm in rear the cardiac mesoderm. Stromal cells are isolated from the notochord before the 13-somite stage (that is before day (E) 8.5 in mice or the equivalent in human development) or the notochord or portions thereof from the same or a related species may be placed in juxta position with primitive pre-pancreatic endoderm or with ES. ICM edc cells from which such primates endodermals cells originate. This differentiation may be enhanced by the exogenous administration of growth factors and cytokines that direct the differentiation of the endothelial cells including but not limited to growth hormone, prolactin, placental lactogen, IGF-1 and IGF-II, gastrin, glucagon-like peptide (GLP-1), exendin, EGF, betacellulin, activin A, activin B, HGF-SF, PDGF, FGF-2,7, Reg protein, parathyroid hormone related peptide (PTH&P), NGF, Ep-CAM, laminin, nicotinamide, or coding sequences for the above where they are peptides, administered to the stem cells or the inducing cells.

After obtaining insulin expressing cells, purification may be obtained through the use of genetic skeleton where a B-cell specific promoter and selectable maker are transfected and used to purify or the use of a selectable marker using the endogenous B-cell specific promoter.

The third step involves. The B-cells are cultured for 2 weeks-4 months, preferably >2 months to mature them into transplantable cells capable of regulating glucone cultured in standard conditions maturation through with normal physiological glucose.

The production of B-cells by the instant invention has the advantage that human ES can be genetically modified to prevent autoimmune destruction. Or alternatively, the patients somatic cells (fibroblasts) may be so genetically modified and then used as nuclear donors in NT to produce cloned ICM, ES cells, or other pluripotent stem cells that can be differentiated into B-cells that have improved suitability in autoimmune diabetes (e.g. Type I diabetes) such genetic modifications include but are not limited to the modulation of MHC Class-I expression, blocking cytokine reception signaling pathways, or expressing inhibitory cytokines (the latter two examples could be applied to hematopoietic stem cells as in the above example. The HSC example and the HSC grafted in the patient in paralleled with the B-cell graft. In addition, the B-cells produced in this invention may be engineered to express increased levels of cytoprotective genes such as antiapoptotic proteins, heat stock proteins and anti-oxidant enzymes such as superoxide slismatase and catalase.

Other variations of the invention may be envisioned by the skilled artisan upon reading the disclosure, and are included in the invention to the extent that they are encompassed within the scope of the appended claims.

What is claimed:

1. A method of producing differentiated mammalian cells or tissues, comprising:
(a) obtaining an inner cell mass or a pluripotent or multipotent stem cell;
(b) mixing said inner cell mass or portion thereof or pluripotent or multipotent stem cell with developing allogeneic or xenogeneic cells; and
(c) implanting or injecting said mixture of cells into a suitable host embryo, fetus or animal so as to generate differentiated mammalian cells or tissues.

2. A method of producing differentiated mammalian cells or tissues comprising:
(a) obtaining a blastocyst, morula inner cell mass, or portion thereof or pluripotent or multipotent mammalian stem cell;
(b) mixing said blastocyst, morula inner cell mass or portion thereof or pluripotent or multipotent stem cell with a developing or differentiated allogeneic or xenogeneic cell; and
(c) culturing said cell mixture under conditions that promote development of a desired differentiated cell type.

3. The method of claim 1 or 2, wherein said differentiated mammalian cells or tissues are human cells or tissues.

4. The method of claim 1 or 2, wherein said differentiated cells or tissues are replacement cells or tissues generated for a mammal in need thereof.

5. The method of claim 4, wherein said replacement cells or tissues have the same nuclear genotype as the mammal in need thereof.

6. The method of claim 1 or 2, wherein said inner cell mass or pluripotent or multipotent stem cell is isolated following nuclear transfer using a donor cell or cell nucleus from said mammal in need of said replacement cells or tissues.

7. The method of claim 1 or 2 wherein the cells in step (b) are produced from an embryo produced by parthenogenesis.

8. The method of claim 7 where said embryo is produced by parthenogenic activation of an unfertilized ovum.

9. The method of claim 1 or 2 wherein the cells in step (b) are obtained from an embryo produced by IVF.

10. The method of claim 1 or 2 wherein said pluripotent or multipotent cell is obtained from a CICM culture.

11. The method of claim 1 or 2, wherein said inner cell mass or pluripotent or multipotent stem cell is an embryonic or adult cell.

12. The method of claim 1 or 2, wherein said inner cell mass or pluripotent or multipotent stem cell is an embryonic cell selected from the group consisting of primordial germ cells, embryoid body cells, ES cells, ICM cells, blastocyst cells, committed progenitor cells, mesenchymal stem cells (MSC), neural crest cells, cranial crest cells.

13. The method of claim 12 wherein said cells are produced by nuclear transfer IVF, pathenogencis or transfer of cytoplasm of embryonic cells into another cell.

14. The method of claim 1 or 2, wherein said pluripotent or multipotent stem cell is an adult stem cell selected from the group consisting of mesenchymal stem cells (MSC), hematopoietic stem cells, stromal stem cells, neural precursor cells, liver precursor cells, skin precursor cells, mesodermal precursor cells, endodermal precursor cells, ectodermal precursor cells.

15. The method of claim 1 or 2, wherein said replacement cells or tissues are selected from the group consisting of pancreatic islet cells, liver cells, kidney cells, lung cells, gut organ tissues, heart muscle cells or other cardiac and vascular tissue, skin cells and other fibroblasts, muscle cells, cells of sensory organs such as the eyes, nose, tongue, ears, hematopoietic cells and cells of the lymph and immune systems, skeletal and cartilage cells, neural cells and tissues, reproduction and endocrine gland cells and tissues.

16. The method of claim 1, wherein said developing or developed allogeneic or xenogeneic cells are a mixture of different cells.

17. The method of claim 1 or 2 wherein said developing or developed allogeneic or xenogeneic cells comprises endothelial inducer cells obtained from the developing or mature tissue type that is to be produced in vivo or in vitro.

18. The method of claim 2 wherein the developing allogeneic or xenogeneic cell used to promote differentiation comprises a stromal inducer.

19. The method of claim 15, wherein said mixture of cells comprises cells from more than one germ layer.

20. The method of claim 1 or 2, wherein said allogeneic or xenogeneic cells are animal teratoma or teratocarcinoma cells.

21. The method of claim 1 or 2, wherein said allogeneic or xenogeneic cells are animal embryonic or fetal cells.

22. The method of claim 21, wherein said allogeneic or xenogeneic animal embryonic or fetal cells are dissociated or form part of an intact embryo, fetus, embryonic structure or fetal organ or section thereof.

23. The method of claim 21, wherein said allogeneic or xenogeneic embryonic or fetal cells are cells from a NT embryo, parthenogenic embryo, IVF embryo or CICM culture.

24. The method of claim 23, wherein said allogeneic or xenogeneic embryonic or fetal cells of are further mixed with a hormone, cytokine, growth factor or other accessory factor.

25. The method of claim 1 or 2, wherein said mixture of cells is aggregated with a biocompatible carrier material prior to being implanted into said suitable host embryo, fetus or animal.

26. The method of claim 25, wherein said biocompatible carrier is introduce into the cell mixture of (b) and this mixture cultured in a tissue culture apparatus.

27. The method of claim 25, wherein said carrier material is selected from the group consisting of proteins such as collagen, gelatin, fibrin/fibrin clots, demineralized bone matrix (DBM), Matrigel® and Collastat®; carbohydrates such as starch, polysaccharides, saccharides, amylopectin, Hetastarch, alginate, methylcellulose and carboxymethylcellulose; proteoglycans, such as hyaluronate; agar; synthetic polymers, including polyesters, especially of normal metabolites such as glycolic acid, lactic acid, caprolactone, maleic acid, and glycols, polyethylene glycol, polyhydroxyethylmethacrylate, polymethylmethacrylate, polyamino acids, polydioxanone, and polyanhydrides; ceramics, such as tricalcium phosphate, hydroxyapatite, alumina, zirconia, bone mineral and gypsum; glasses such as Bioglass, A-W glass, and calcium phosphate glasses; metals including titanium, Ti-6Al-4V, cobalt-chromium alloys, stainless steel and tantalum; and hydrogel matrices.

28. The method of claim 1 or 2, wherein said suitable host embryo, fetus or animal is selected from the group consisting of mice, rats, sheep, pigs, cows.

29. The method of claim 28, wherein said suitable host fetus or animal is immuno-compromised, immuno-suppressed or tolerized.

30. The method of claim 29, wherein said suitable host fetus or animal is tolerized by exposure to antigens, cells or tissues prior to the development of self-recognition.

31. The method of claim 30, wherein said mixture of cells is implanted or injected into the thymus, lungs, muscle wall, liver, heart, brain, pancreas, kidney, of said host fetus or animal.

32. The method of claim 28, wherein said mixture of cells is implanted or injected into a suitable host embryo.

33. The method of claim 32, wherein said mixture of cells is implanted or injected into the endoderm, mesoderm or ectoderm of said suitable host embryo, or into specific regions derived therefrom.

34. The method of claim 33, wherein said mixture of cells is implanted or injected into the ectoderm of the host embryo, or into specific regions derived therefrom.

35. The method of claim 34, wherein said mixture of cells is implanted or injected into the general body ectoderm, the neural plate, the neural crest or the ectodermal placodes of said ectoderm, or into specific regions derived therefrom.

36. The method of claim 33, wherein said mixture of cells is implanted or injected into the mesoderm of the host embryo, or into specific regions derived therefrom.

37. The method of claim 36, wherein said mixture of cells is implanted or injected into the paraxial mesoderm, the intermediate mesoderm or the lateral plate, or into specific regions derived therefrom.

38. The method of claim 29, wherein said mixture of cells is implanted or injected following segmentation of the paraxial mesoderm into a mesodermal somite, or into specific regions derived therefrom.

39. The method of claim 29, wherein said mixture of cells is implanted or injected following division of the lateral plate mesoderm into the intraembryonic splanchnopleure, or into specific regions derived therefrom.

40. The method of claim 21, wherein said host animal is a SCID or nude mouse.

41. The method of claim 21, wherein said mixture of cells is implanted or injected under the kidney capsule or into the peritoneum of said host animal.

42. A method of obtaining differentiated mammalian cells or tissues, comprising:
(a) obtaining a inner cell mass or pluripotent or multipotent stem cell;
(b) mixing said inner cell mass or portion thereof or pluripotent or multipotent stem cell with developing or developed allogeneic or xenogeneic cells;
(c) implanting or injecting said mixture of cells into a suitable host embryo, fetus or animal or culturing said mixture of cell in vitro so as to generate differentiated mammalian cells or tissues; and
(d) obtaining said differentiated mammalian cells or tissues.

43. The method of claim 42, wherein said differentiated cells or tissues are isolated by virtue of a selectable marker.

44. The method of claim 43, wherein said selectable marker is expressed from a heterologous DNA construct.

45. The method of claim 44, wherein said selection is commenced during in vivo development.

46. The method of claim 42, wherein said differentiated cells or tissues are isolated using immunoaffinity purification or FACS.

47. The method of claim 42, wherein said inner cell mass or pluripotent or multipotent stem cell is genetically engineered by inserting, deleting or modifying a gene or other genetic material prior to mixture with said allogeneic or xenogeneic cells.

48. The differentiated cells or tissues produced by the method of claim 42.

49. A method of treating a patient in need of replacement cells or tissues by transplanting into said patient the cells or tissues produced by the method of claim 42.

50. A chimeric mixture or structure of cells, comprising
(a) at least one pluripotent or multipotent stem cell; and
(b) allogeneic or xenogeneic cells and/or tissues, wherein said mixture facilitates differentiation of said pluripotent or multipotent stem cell along a particular developmental path.

51. The chimeric mixture or structure of claim 50, wherein said mixture is further implanted into an in vivo environment in order to facilitate differentiation of said pluripotent or multipotent stem cell.

52. The chimeric mixture or structure of claim 51, wherein said mixture is designed to facilitate the differentiation of a pluripotent or multipotent stem cell into a cell selected from the group consisting of pancreatic islet cells, liver cells, kidney cells, lung cells, gut organ tissues, heart muscle cells or other cardiac and vascular tissue, skin cells and other fibroblasts, muscle cells, cells of sensory organs such as the eyes, nose, tongue, ears, hematopoietic cells and cells of the lymph and immune systems, skeletal and cartilage cells, neural cells and tissues, reproduction and endocrine gland cells and tissues.

53. The chimeric mixture or structure of claim 52, wherein said mixture is designed to facilitate the differentiation of a pluripotent or multipotent cell into a pancreatic islet cell.

54. The chimeric mixture or structure of claim 41, wherein said pluripotent or multipotent stem cell is an embryonic cell selected from the group consisting of primordial germ cells, embryoid body cells, ES cells, ICM cells, blastocyst cells, committed progenitor cells, mesenchymal stem cells (MSC), neural crest cells, cranial crest cells.

55. The chimeric mixture or structure of claim 41, wherein said pluripotent or multipotent stem cell is an adult stem cell selected from the group consisting of mesenchymal stem cells (MSC), hematopoietic stem cells, stromal stem cells, neural precursor cells, liver precursor cells, skin precursor cells, mesodermal precursor cells, endodermal precursor cells, ectodermal precursor cells.

56. The chimeric mixture or structure of claim 55, wherein said pluripotent cell is an ICM cell.

57. The chimeric mixture or structure of claim 56, wherein said ICM cell was obtained using nuclear transfer.

58. The chimeric mixture or structure of claim 56, wherein said ICM cell was obtained using nuclear transfer from a human donor cell.

59. The chimeric mixture or structure of claim 49, wherein said at least one pluripotent or multipotent stem cell is genetically engineered by inserting, deleting or modifying a gene or other genetic material prior to mixture with said allogeneic or xenogeneic cells.

60. The chimeric mixture or structure of claim 58, wherein said human donor cell is genetically engineered by inserting, deleting or modifying a gene or other genetic material prior to nuclear transfer.

61. The chimeric mixture or structure of claim 50 further comprising a carrier material is selected from the group consisting of proteins such as collagen, gelatin, fibrin/fibrin clots, demineralized bone matrix (DBM), Matrigel® and Collastat®; carbohydrates such as starch, polysaccharides, saccharides, amylopectin, Hetastarch, alginate, methylcellulose and carboxymethylcellulose; proteoglycans, such as hyaluronate; agar; synthetic polymers, including polyesters, especially of normal metabolites such as glycolic acid, lactic acid, caprolactone, maleic acid, and glycols, polyethylene glycol, polyhydroxyethylmethacrylate, polymethylmethacrylate, polyamino acids, polydioxanone, and polyanhydrides; ceramics, such as tricalcium phosphate, hydroxyapatite, alumina, zirconia, bone mineral and gypsum; glasses such as Bioglass, A-W glass, and calcium phosphate glasses; metals including titanium, Ti-6Al-4V, cobalt-chromium alloys, stainless steel and tantalum; and hydrogel matrices.
